(19) United States
(12) Patent Application Publication
(10) Pub. No.: US 2020/0299772 A1
(43) Pub. Date: Sep. 24, 2020

(54) ASSESSING AND TREATING SPINAL MUSCULAR ATROPHY

(71) Applicant: Mayo Foundation for Medical Education and Research, Rochester, MN (US)

(72) Inventors: Devin Oglesbee, Rochester, MN (US); Noemi Vidal Folch, Rochester, MN (US)

(73) Assignee: Mayo Foundation for Medical Education and Research, Rochester, MN (US)

(21) Appl. No.: 16/825,775

(22) Filed: Mar. 20, 2020

Related U.S. Application Data
(60) Provisional application No. 62/821,845, filed on Mar. 21, 2019.

(51) Int. Cl. C12Q 1/6883; C12Q 1/6818; C12Q 1/6827; C12Q 1/686

(52) U.S. Cl. CPC C12Q 1/6883; C12Q 1/6818; C12Q 2600/156; C12Q 1/686; C12Q 1/6827

(57) ABSTRACT

This document relates to methods and materials for assessing and/or treating a mammal (e.g., a human) having, or at risk of developing, a spinal condition (e.g., spinal muscular atrophy (SMA)). In some cases, a mammal can be identified as having, or as being likely to develop, a spinal condition (e.g., SMA), and, optionally, can be treated. For example, a mammal can be identified as having, or as being likely to develop, a spinal condition (e.g., SMA), based, at least in part, on the modification of nucleic acid that can encode a survival motor neuron (SMN) polypeptide (e.g., homozygous deletion of exon 7 of SMN1 nucleic acid encoding a SMN polypeptide and the genomic copy number of SMN2 nucleic acid encoding a SMN polypeptide) in a sample from a mammal and, optionally, can be treated.

### CROSS-REFERENCE TO RELATED APPLICATIONS

This application claims the benefit of U.S. Provisional Application Ser. No. 62/821,845, filed Mar. 21, 2019. The disclosure of the prior application is considered part of (and is incorporated by reference in) the disclosure of this application.

### BACKGROUND

### 1. Technical Field

This document relates to methods and materials for assessing and/or treating a mammal (e.g., a human) having, or at risk of developing, a spinal condition (e.g., spinal muscular atrophy (SMA)). In some cases, a mammal can be identified as having, or as being likely to develop, a spinal condition (e.g., SMA), and, optionally, can be treated. For example, a mammal can be identified as having, or as being likely to develop, a spinal condition (e.g., SMA), based, at least in part, on the modification of nucleic acid that can encode a survival motor neuron (SMN) polypeptide (e.g., homozygous deletion of exon 7 of SMN1 nucleic acid encoding a SMN polypeptide and the genomic copy number of SMN2 nucleic acid encoding a SMN polypeptide) in a sample from a mammal and, optionally, can be treated.

### 2. Background Information

SMA is an autosomal recessive neuromuscular disorder characterized by degeneration of motor neurons in the spinal cord and leading to muscular atrophy and respiratory failure. A diploid mammal such as a human has two copies of each gene present in its genome. SMA is most commonly caused by a homozygous deletion of exon 7 in the SMN1 gene (e.g., a deletion of exon 7 that is present in both copies of the SMN1 gene). In humans, a nearly identical copy gene, SMN2, is also present, and SMN2 copy number is predictive of disease severity. SMA newborn screening (NB S) adoption is increasing since the Health and Human Services (HHS) Secretary endorsed the inclusion of SMA into a Recommended Uniform Screening Panel. However, there is a lack of standardized NBS methods. Carrier screening for SMA is recommended by both the American College of Medical Genetics and Genomics (ACMG) and the American College of Obstetricians and Gynecologists (ACOG) and is routinely offered to women, regardless of race or ethnicity, before conception or early in pregnancy.

### SUMMARY

The loss of motor neurons in humans with SMA is irreversible, and early treatment is useful to modulate the rapid and progressive degeneration seen in SMA, especially in type 1 SMA. Further, diagnostic delay can be very common in SMA, and most patients have progressed past the point where maximal benefit is achievable before therapeutic interventions occur.

This document provides methods and materials for using a sensitive and specific method (e.g., multiplex digital PCR (dPCR) such as droplet digital PCR (ddPCR)) for assessing and/or treating a mammal (e.g., a human) having, or at risk of developing, a spinal condition (e.g., SMA). In some cases, a mammal can be identified as having, or as being likely to develop, a spinal condition (e.g., SMA) based, at least in part, on the modification of nucleic acid encoding a SMN polypeptide (e.g., homozygous deletion of exon 7 of SMN1 nucleic acid encoding a SMN polypeptide and the genomic copy number of SMN2 nucleic acid encoding a SMN polypeptide) in a sample from a mammal and, optionally, can be treated. For example, the presence of a homozygous deletion of exon 7 of SMN1 nucleic acid encoding a SMN polypeptide can be used to identify a mammal as having, or as being at risk of developing, a spinal condition (e.g., SMA), and the genomic copy number of SMN2 nucleic acid encoding a SMN polypeptide can be used to predict the severity of the spinal condition (e.g., fewer genomic copies of SMN2 nucleic acid encoding a SMN polypeptide can indicate that a spinal condition such as SMA is likely to be more severe, while more genomic copies of SMN2 nucleic acid can indicate that a spinal condition such as SMA is likely to be less severe). In some cases, multiplex dPCR (e.g., multiplex dPCR such as high-throughput multiplex ddPCR) can be used to simultaneously determine homozygous deletion of exon 7 of SMN1 nucleic acid encoding a SMN polypeptide and the genomic copy number of SMN2 nucleic acid encoding a SMN polypeptide in a sample from a mammal.

As demonstrated herein, multiplex ddPCR can be used to simultaneously detect homozygous deletion of exon 7 of SMN1 nucleic acid encoding a SMN polypeptide and the genomic copy number of SMN2 nucleic acid encoding a SMN polypeptide in genomic DNA from dried blood spots, whole blood, and cultured cells. Healthy patients showed 1 to 5 copies of exon 7 of SMN1 nucleic acid encoding a SMN polypeptide, and SMA patients showed 0 copies of exon 7 of SMN1 nucleic acid encoding a SMN polypeptide. Samples from SMA confirmed positives were identified using the multiplex ddPCR method with a sensitivity of 100% and a specificity of 100%.

Having the ability to use a highly sensitive and highly specific multiplex dPCR method to determine the presence or absence of homozygous deletion of exon 7 in a genomic SMN1 nucleic acid encoding a SMN polypeptide and to determine the genomic copy number of SMN2 nucleic acid encoding a SMN polypeptide in a sample from a human (e.g., a newborn human) can allow humans to be identified as having, as being likely to develop, or as being a carrier of SMA using a single, cost-effective assay that can be used for early (e.g., newborn) screening and early diagnosis of SMA. Further, early screening and diagnosis of SMA provides the opportunity to administer early treatment (e.g., before significant motor neuron loss occurs) to a human (e.g., a newborn human) identified as having, or as being at risk of developing, SMA.

In general, one aspect of this document features methods for detecting homozygous deletion of exon 7 in a genomic SMN1 nucleic acid encoding a SMN polypeptide and to determine the genomic copy number of SMN2 nucleic acid encoding a SMN polypeptide in genomic DNA of a sample from a human. The methods can include, or consist essentially of, subjecting genomic DNA from the mammal to ddPCR where the ddPCR includes contacting the genomic DNA with (a) a first oligonucleotide primer pair for amplification of exon 7 of a SMN1 nucleic acid encoding a SMN polypeptide, where the first oligonucleotide primer pair is capable of generating a SMN1 exon 7 amplicon during the ddPCR, and (b) a first probe capable of hybridizing to the SMN1 exon 7 amplicon, where the first probe is a locked nucleic acid (LNA) oligonucleotide probe, and where the first probe comprises a first fluorescent label and at least one fluorescent quencher; and contacting the genomic DNA with (a) a second oligonucleotide primer pair for amplification of SMN2 nucleic acid encoding a SMN polypeptide, where the second oligonucleotide primer pair is capable of generating a SMN2 amplicon, and (b) a second probe that can hybridize to the SMN2 amplicon, where the second probe is a LNA oligonucleotide probe, where the probe comprises a second fluorescent label and at least one fluorescent quencher; where the first fluorescent label and the second fluorescent label are different fluorescent labels; and detecting a first fluorescent signal emitted when the first probe is hybridized to the SMN1 exon 7 amplicon, and detecting a second fluorescent signal emitted when the second probe is hybridized to the SMN2 amplicon. The mammal can be a human. The mammal can be a newborn mammal. The mammal can be a prenatal mammal. The sample can be a blood sample (e.g., a dried blood sample (DBS)). The first oligonucleotide primer pair can include an oligonucleotide primer including the nucleotide sequence set forth in SEQ ID NO:1 and an oligonucleotide primer including the nucleotide sequence set forth in SEQ ID NO:2. The first oligonucleotide primer pair can include an oligonucleotide primer including the nucleotide sequence set forth in SEQ ID NO:10 and an oligonucleotide primer including the nucleotide sequence set forth in SEQ ID NO:11. The first oligonucleotide primer pair can include an oligonucleotide primer including the nucleotide sequence set forth in SEQ ID NO:13 and an oligonucleotide primer including the nucleotide sequence set forth in SEQ ID NO:14. The first probe can include the nucleotide sequence set forth in SEQ ID NO:3, an internal quencher, and a terminal quencher on a 3′ end of the first probe, where the first fluorescent label is on a 5′ end of the first probe. The second oligonucleotide primer pair can include an oligonucleotide primer including the nucleotide sequence set forth in SEQ ID NO:4 and an oligonucleotide primer including the nucleotide sequence set forth in SEQ ID NO:5. The second probe can include the nucleotide sequence set forth in SEQ ID NO:6, an internal quencher, and a terminal quencher on a 3′ end of the second probe, where the second fluorescent label is on a 5′ end of the second probe. The first fluorescent label can be a fluorescein amidite (FAM), and the second fluorescent label can be a hexachloro-fluorescein (HEX). The ddPCR also can include contacting the genomic DNA with (a) a third oligonucleotide primer pair for amplification of nucleic acid encoding a reference polypeptide, where the third oligonucleotide primer pair is capable of generating a reference amplicon, and (b) a third probe that can hybridize to the reference amplicon, where the third probe is a LNA oligonucleotide probe, where the third probe comprises a third fluorescent label and at least one fluorescent quencher; where the third fluorescent label is different from the first fluorescent label and from the second fluorescent label; and detecting a third fluorescent signal emitted when the third probe is hybridized to the reference amplicon. The third oligonucleotide primer pair can include an oligonucleotide primer including the nucleotide sequence set forth in SEQ ID NO:7 and an oligonucleotide primer including the nucleotide sequence set forth in SEQ ID NO:8. The third probe can include the nucleotide sequence set forth in SEQ ID NO:9, an internal quencher, and a terminal quencher on a 3′ end of the third probe, where the third fluorescent label is on a 5′ end of the third probe. The third fluorescent label can be a HEX.

In another aspect, this document features methods for treating a mammal having, or at risk of developing, spinal muscular atrophy (SMA) where the methods can include, or consist essentially of, detecting homozygous deletion of exon 7 in a genomic SMN1 nucleic acid encoding a SMN polypeptide and to determine the genomic copy number of SMN2 nucleic acid encoding a SMN polypeptide in genomic DNA in a sample from a human, where the detecting comprises subjecting genomic DNA from the mammal to ddPCR, where the ddPCR comprises contacting the genomic DNA with (a) a first oligonucleotide primer pair for amplification of exon 7 of a SMN1 nucleic acid encoding a SMN polypeptide, where the first oligonucleotide primer pair is capable of generating a SMN1 exon 7 amplicon, and (b) a first probe that can hybridize to the SMN1 exon 7 amplicon, where the first probe is a locked nucleic acid (LNA) oligonucleotide probe, and where the first probe comprises a first fluorescent label and at least one fluorescent quencher; and contacting the genomic DNA with (a) a second oligonucleotide primer pair for amplification of SMN2 nucleic acid encoding a SMN polypeptide, where the second oligonucleotide primer pair is capable of generating a SMN2 amplicon, and (b) a second probe that can hybridize to the SMN2 amplicon, where the second probe is a LNA oligonucleotide probe, where the probe comprises a second fluorescent label and at least one fluorescent quencher; where the first fluorescent label and the second fluorescent label are different fluorescent labels; and detecting a first fluorescent signal emitted when the first probe is hybridized to the SMN1 exon 7 amplicon, and detecting a second fluorescent signal emitted when the second probe is hybridized to the SMN2 amplicon; and when homozygous deletion of exon 7 in a genomic SMN1 nucleic acid encoding a SMN polypeptide is detected and when the genomic copy number of SMN2 nucleic acid encoding a SMN polypeptide is 1 or 2, administering one or more SMA treatments to the mammal. The SMA treatment can be administration of an agent that can increase exon 7 inclusion in SMN2 messenger ribonucleic acid (mRNA), occupational therapy, physical therapy, or speech and language therapy. The mammal can be a human. The mammal can be a newborn mammal. The mammal can be a prenatal mammal. The sample can be a blood sample (e.g., DBS). The first oligonucleotide primer pair can include an oligonucleotide primer including the nucleotide sequence set forth in SEQ ID NO:1 and an oligonucleotide primer including the nucleotide sequence set forth in SEQ ID NO:2. The first oligonucleotide primer pair can include an oligonucleotide primer including the nucleotide sequence set forth in SEQ ID NO:10 and an oligonucleotide primer including the nucleotide sequence set forth in SEQ ID NO:11. The first oligonucleotide primer pair can include an oligonucleotide primer including the nucleotide sequence set forth in SEQ ID NO:13 and an oligonucleotide primer including the nucleotide sequence set forth in SEQ ID NO:14. The first probe can include the nucleotide sequence set forth in SEQ ID NO:3, an internal quencher, and a terminal quencher on a 3′ end of the first probe, where the first fluorescent label is on a 5′ end of the first probe. The second oligonucleotide primer pair can include an oligonucleotide primer including the nucleotide sequence set forth in SEQ ID NO:4 and an oligonucleotide primer including the nucleotide sequence set forth in SEQ ID NO:5. The second probe can include the nucleotide sequence set forth in SEQ ID NO:6, an internal quencher, and a terminal quencher on a 3′ end of the second probe, and where the second fluorescent label is on a 5′ end of the second probe. The first fluorescent label can be a FAM, and the second fluorescent label can be a HEX. The ddPCR also can include contacting the genomic DNA with (a) a third oligonucleotide primer pair for amplification of nucleic acid encoding a reference polypeptide, where the third oligonucleotide primer pair is capable of generating a reference amplicon, and (b) a third probe that can hybridize to the reference amplicon, where the third probe is a LNA oligonucleotide probe, where the third probe comprises a third fluorescent label and at least one fluorescent quencher; where the third fluorescent label is different from the first fluorescent label and from the second fluorescent label; and detecting a third fluorescent signal emitted when the third probe is hybridized to the reference amplicon. The third oligonucleotide primer pair can include an oligonucleotide primer including the nucleotide sequence set forth in SEQ ID NO:7 and an oligonucleotide primer including the nucleotide sequence set forth in SEQ ID NO:8. The method of any one of claims 30-31, where the third probe includes the nucleotide sequence set forth in SEQ ID NO:9, an internal quencher, and a terminal quencher on a 3′ end of the third probe, and where the third fluorescent label is on a 5′ end of the third probe. The third fluorescent label can be a HEX.

In another aspect, this document features methods for treating a mammal having, or at risk of developing, SMA where the methods can include, or consist essentially of, detecting homozygous deletion of exon 7 in a genomic SMN1 nucleic acid encoding a SMN polypeptide and detecting a genomic copy number of SMN2 nucleic acid encoding a SMN polypeptide in genomic DNA in a sample from a human, where the detecting comprises subjecting genomic DNA from the mammal to ddPCR, where the ddPCR comprises contacting the genomic DNA with (a) a first oligonucleotide primer pair for amplification of exon 7 of a SMN1 nucleic acid encoding a SMN polypeptide, where the first oligonucleotide primer pair is capable of generating a SMN1 exon 7 amplicon, and (b) a first probe that can hybridize to the SMN1 exon 7 amplicon, where the first probe is a locked nucleic acid (LNA) oligonucleotide probe, and where the first probe comprises a first fluorescent label and at least one fluorescent quencher; and contacting the genomic DNA with (a) a second oligonucleotide primer pair for amplification of SMN2 nucleic acid encoding a SMN polypeptide, where the second oligonucleotide primer pair is capable of generating a SMN2 amplicon, and (b) a second probe that can hybridize to the SMN2 amplicon, where the second probe is a LNA oligonucleotide probe, where the probe comprises a second fluorescent label and at least one fluorescent quencher; where the first fluorescent label and the second fluorescent label are different fluorescent labels; and detecting a first fluorescent signal emitted when the first probe is hybridized to the SMN1 exon 7 amplicon, and detecting a second fluorescent signal emitted when the second probe is hybridized to the SMN2 amplicon; and when homozygous deletion of exon 7 in a genomic SMN1 nucleic acid encoding a SMN polypeptide is detected and when the genomic copy number of SMN2 nucleic acid encoding a SMN polypeptide is 3, 4, or 5, selecting the mammal for monitoring for symptoms of SMA. The monitoring can include physical examinations to test gross motor abilities, electromyography (EMG) to measure a muscle's ability to respond to electrical stimulation, and muscle biopsy tests to examine muscle for signs of muscle degeneration. The mammal can be a human. The mammal can be a newborn mammal. The mammal can be a prenatal mammal. The sample can be a blood sample (e.g., DBS). The first oligonucleotide primer pair can include an oligonucleotide primer including the nucleotide sequence set forth in SEQ ID NO:1 and an oligonucleotide primer including the nucleotide sequence set forth in SEQ ID NO:2. The first oligonucleotide primer pair can include an oligonucleotide primer including the nucleotide sequence set forth in SEQ ID NO:10 and an oligonucleotide primer including the nucleotide sequence set forth in SEQ ID NO:11. The first oligonucleotide primer pair can include an oligonucleotide primer including the nucleotide sequence set forth in SEQ ID NO:13 and an oligonucleotide primer including the nucleotide sequence set forth in SEQ ID NO:14. The first probe can include the nucleotide sequence set forth in SEQ ID NO:3, an internal quencher, and a terminal quencher on a 3′ end of the first probe, where the first fluorescent label is on a 5′ end of the first probe. The second oligonucleotide primer pair can include an oligonucleotide primer including the nucleotide sequence set forth in SEQ ID NO:4 and an oligonucleotide primer including the nucleotide sequence set forth in SEQ ID NO:5. The second probe can include the nucleotide sequence set forth in SEQ ID NO:6, an internal quencher, and a terminal quencher on a 3′ end of the second probe, and where the second fluorescent label is on a 5′ end of the second probe. The first fluorescent label can be a FAM, and the second fluorescent label can be a HEX. The ddPCR also can include contacting the genomic DNA with (a) a third oligonucleotide primer pair for amplification of nucleic acid encoding a reference polypeptide, where the third oligonucleotide primer pair is capable of generating a reference amplicon, and (b) a third probe that can hybridize to the reference amplicon, where the third probe is a LNA oligonucleotide probe, where the third probe comprises a third fluorescent label and at least one fluorescent quencher; where the third fluorescent label is different from the first fluorescent label and from the second fluorescent label; and detecting a third fluorescent signal emitted when the third probe is hybridized to the reference amplicon. The third oligonucleotide primer pair can include an oligonucleotide primer including the nucleotide sequence set forth in SEQ ID NO:7 and an oligonucleotide primer including the nucleotide sequence set forth in SEQ ID NO:8. The method of any one of claims 30-31, where the third probe includes the nucleotide sequence set forth in SEQ ID NO:9, an internal quencher, and a terminal quencher on a 3′ end of the third probe, and where the third fluorescent label is on a 5′ end of the third probe. The third fluorescent label can be a HEX.

In another aspect, this document features methods for treating a mammal having, or at risk of developing, SMA where the methods can include, or consist essentially of, administering one or more SMA treatments to a mammal identified as having SMA by detecting homozygous deletion of exon 7 in a genomic SMN1 nucleic acid encoding a SMN polypeptide and detecting the genomic copy number of SMN2 nucleic acid encoding a SMN polypeptide in genomic DNA in a sample from a human, where the detecting comprises subjecting genomic DNA from the mammal to ddPCR, where the ddPCR comprises contacting the genomic DNA with (a) a first oligonucleotide primer pair for amplification of exon 7 of a SMN1 nucleic acid encoding a SMN polypeptide, where the first oligonucleotide primer pair is capable of generating a SMN1 exon 7 amplicon, and (b) a first probe that can hybridize to the SMN1 exon 7 amplicon, where the first probe is a locked nucleic acid (LNA) oligonucleotide probe, and where the first probe comprises a first fluorescent label and at least one fluorescent quencher; and contacting the genomic DNA with (a) a second oligonucleotide primer pair for amplification of SMN2 nucleic acid encoding a SMN polypeptide, where the second oligonucleotide primer pair is capable of generating a SMN2 amplicon, and (b) a second probe that can hybridize to the SMN2 amplicon, where the second probe is a LNA oligonucleotide probe, where the probe comprises a second fluorescent label and at least one fluorescent quencher; where the first fluorescent label and the second fluorescent label are different fluorescent labels; and detecting a first fluorescent signal emitted when the first probe is hybridized to the SMN1 exon 7 amplicon, and detecting a second fluorescent signal emitted when the second probe is hybridized to the SMN2 amplicon; and when homozygous deletion of exon 7 in a genomic SMN1 nucleic acid encoding a SMN polypeptide is detected and when the genomic copy number of SMN2 nucleic acid encoding a SMN polypeptide is 1 or 2. The SMA treatment can be administration of an agent that can increase exon 7 inclusion in SMN2 messenger ribonucleic acid (mRNA), occupational therapy, physical therapy, or speech and language therapy. The mammal can be a human. The mammal can be a newborn mammal. The mammal can be a prenatal mammal. The sample can be a blood sample (e.g., DBS). The first oligonucleotide primer pair can include an oligonucleotide primer including the nucleotide sequence set forth in SEQ ID NO:1 and an oligonucleotide primer including the nucleotide sequence set forth in SEQ ID NO:2. The first oligonucleotide primer pair can include an oligonucleotide primer including the nucleotide sequence set forth in SEQ ID NO:10 and an oligonucleotide primer including the nucleotide sequence set forth in SEQ ID NO:11. The first oligonucleotide primer pair can include an oligonucleotide primer including the nucleotide sequence set forth in SEQ ID NO:13 and an oligonucleotide primer including the nucleotide sequence set forth in SEQ ID NO:14. The first probe can include the nucleotide sequence set forth in SEQ ID NO:3, an internal quencher, and a terminal quencher on a 3′ end of the first probe, where the first fluorescent label is on a 5′ end of the first probe. The second oligonucleotide primer pair can include an oligonucleotide primer including the nucleotide sequence set forth in SEQ ID NO:4 and an oligonucleotide primer including the nucleotide sequence set forth in SEQ ID NO:5. The second probe can include the nucleotide sequence set forth in SEQ ID NO:6, an internal quencher, and a terminal quencher on a 3′ end of the second probe, and where the second fluorescent label is on a 5′ end of the second probe. The first fluorescent label can be a FAM, and the second fluorescent label can be a HEX. The ddPCR also can include contacting the genomic DNA with (a) a third oligonucleotide primer pair for amplification of nucleic acid encoding a reference polypeptide, where the third oligonucleotide primer pair is capable of generating a reference amplicon, and (b) a third probe that can hybridize to the reference amplicon, where the third probe is a LNA oligonucleotide probe, where the third probe comprises a third fluorescent label and at least one fluorescent quencher; where the third fluorescent label is different from the first fluorescent label and from the second fluorescent label; and detecting a third fluorescent signal emitted when the third probe is hybridized to the reference amplicon. The third oligonucleotide primer pair can include an oligonucleotide primer including the nucleotide sequence set forth in SEQ ID NO:7 and an oligonucleotide primer including the nucleotide sequence set forth in SEQ ID NO:8. The method of any one of claims 30-31, where the third probe includes the nucleotide sequence set forth in SEQ ID NO:9, an internal quencher, and a terminal quencher on a 3′ end of the third probe, and where the third fluorescent label is on a 5′ end of the third probe. The third fluorescent label can be a HEX.

In another aspect, this document features methods for treating a mammal having, or at risk of developing, SMA where the methods can include, or consist essentially of, selecting a mammal identified as having SMA by detecting homozygous deletion of exon 7 in a genomic SMN1 nucleic acid encoding a SMN polypeptide and detecting a genomic copy number of SMN2 nucleic acid encoding a SMN polypeptide in genomic DNA in a sample from a human for monitoring for symptoms of SMA, where the detecting comprises subjecting genomic DNA from the mammal to ddPCR, where the ddPCR comprises contacting the genomic DNA with (a) a first oligonucleotide primer pair for amplification of exon 7 of a SMN1 nucleic acid encoding a SMN polypeptide, where the first oligonucleotide primer pair is capable of generating a SMN1 exon 7 amplicon, and (b) a first probe that can hybridize to the SMN1 exon 7 amplicon, where the first probe is a locked nucleic acid (LNA) oligonucleotide probe, and where the first probe comprises a first fluorescent label and at least one fluorescent quencher; and contacting the genomic DNA with (a) a second oligonucleotide primer pair for amplification of SMN2 nucleic acid encoding a SMN polypeptide, where the second oligonucleotide primer pair is capable of generating a SMN2 amplicon, and (b) a second probe that can hybridize to the SMN2 amplicon, where the second probe is a LNA oligonucleotide probe, where the probe comprises a second fluorescent label and at least one fluorescent quencher; where the first fluorescent label and the second fluorescent label are different fluorescent labels; and detecting a first fluorescent signal emitted when the first probe is hybridized to the SMN1 exon 7 amplicon, and detecting a second fluorescent signal emitted when the second probe is hybridized to the SMN2 amplicon; and when homozygous deletion of exon 7 in a genomic SMN1 nucleic acid encoding a SMN polypeptide is detected and when the genomic copy number of SMN2 nucleic acid encoding a SMN polypeptide is 3, 4, or 5. The monitoring can include physical examinations to test gross motor abilities, electromyography (EMG) to measure a muscle's ability to respond to electrical stimulation, or muscle biopsy tests to examine muscle for signs of muscle degeneration. The mammal can be a human. The mammal can be a newborn mammal. The mammal can be a prenatal mammal. The sample can be a blood sample (e.g., DBS). The first oligonucleotide primer pair can include an oligonucleotide primer including the nucleotide sequence set forth in SEQ ID NO:1 and an oligonucleotide primer including the nucleotide sequence set forth in SEQ ID NO:2. The first oligonucleotide primer pair can include an oligonucleotide primer including the nucleotide sequence set forth in SEQ ID NO:10 and an oligonucleotide primer including the nucleotide sequence set forth in SEQ ID NO:11. The first oligonucleotide primer pair can include an oligonucleotide primer including the nucleotide sequence set forth in SEQ ID NO:13 and an oligonucleotide primer including the nucleotide sequence set forth in SEQ ID NO:14. The first probe can include the nucleotide sequence set forth in SEQ ID NO:3, an internal quencher, and a terminal quencher on a 3′ end of the first probe, where the first fluorescent label is on a 5′ end of the first probe. The second oligonucleotide primer pair can include an oligonucleotide primer including the nucleotide sequence set forth in SEQ ID NO:4 and an oligonucleotide primer including the nucleotide sequence set forth in SEQ ID NO:5. The second probe can include the nucleotide sequence set forth in SEQ ID NO:6, an internal quencher, and a terminal quencher on a 3′ end of the second probe, and where the second fluorescent label is on a 5′ end of the second probe. The first fluorescent label can be a FAM, and the second fluorescent label can be a HEX. The ddPCR also can include contacting the genomic DNA with (a) a third oligonucleotide primer pair for amplification of nucleic acid encoding a reference polypeptide, where the third oligonucleotide primer pair is capable of generating a reference amplicon, and (b) a third probe that can hybridize to the reference amplicon, where the third probe is a LNA oligonucleotide probe, where the third probe comprises a third fluorescent label and at least one fluorescent quencher; where the third fluorescent label is different from the first fluorescent label and from the second fluorescent label; and detecting a third fluorescent signal emitted when the third probe is hybridized to the reference amplicon. The third oligonucleotide primer pair can include an oligonucleotide primer including the nucleotide sequence set forth in SEQ ID NO:7 and an oligonucleotide primer including the nucleotide sequence set forth in SEQ ID NO:8. The method of any one of claims 30-31, where the third probe includes the nucleotide sequence set forth in SEQ ID NO:9, an internal quencher, and a terminal quencher on a 3′ end of the third probe, and where the third fluorescent label is on a 5′ end of the third probe. The third fluorescent label can be a HEX.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIGS. 1A-1B. Maps of exemplary primers and exemplary probes used for ddPCR are shown in FIG. 1A and FIG. 1B. FIG. 1A. DdPCR primer and probe locations for SMN1 and SMN2 exon 7 copy number assay. Two nucleotide locations, c.739-44G/A and c.744C/T, with uniques differences between SMN1 and SMN2 are shown and were used to enable assay specificity. Locked nucleic acid (LNA) probes are seen as dashed lines and solid arrows indicate DNA primers. FIG. 1B. SMN1 [2+0] allele primer and probe locations. Three nucleotide locations, c.744C/T, g.27134G/T, and c.*3+100A/G, are shown. LNA probes for the SMN1, g.27134T/G nucleotides, and SMN2 c.744T nucleotide are dashed lines. Solid arrows are DNA primers that have specificity for SMN1.

FIGS. 2A-B. The clustering of ddPCR fluorescent droplets as observed by QuantaSoft analysis is shown in FIGS. 2A, 2B, and 2C. Each droplet cluster corresponds to a different population of nucleotide targets measured by the assay. FIG. 2A. DdPCR results from a normal donor, which shows SMN1 exon 7 positive (SMN1+), SMN2 positive (SMN2+), and RPP30 positive (RPP30+) droplet clusters as well as double- and triple-positive droplet clusters. FIG. 2B. Results from an SMA-positive donor sample with a homozygous SMN1 exon 7 deletion showing only SMN2+ and RPP30+ droplet clusters. FIG. 2C. Results from the analysis of a specimen without DNA.

FIG. 3. Distribution of SMN1 and SMN2 exon 7 CNV results in newborn screening dried blood spots. Dot plots show the range of ddPCR CNV levels. Red dots specify the homozygous specimen (SMA positive), blue dots specify the heterozygous specimens (SMA carrier), and yellow dots specify specimens with 2 or more SMN1 CNV (SMA normal). Black dashed lines indicate the thresholds set for interpretation of SMA positive, SMA carrier, and SMA normal.

FIG. 4. Screening and Testing Algorithms for NBS and Carrier Status Determination.

FIGS. 5A-5B. Imprecision of CNV levels at low RPP30 copes for SMN1 CNV (FIG. 5A) and SMN2 CNV (FIG. 5B) are shown. In FIG. 5A, sixteen replicates from 4 different specimens with CNV levels of 0, 1, 2, and 4 SMN1 CNV are plotted. The RPP30 copies for these specimens ranged from 277 to 383 RPP30 copies/well. Dashed red lines indicate the CNV limits that define each CNV level at low RPP30 copies. In FIG. 5B, sixteen replicates from 4 difference specimens with CNV levels of 0, 1, 2, and 3 SMN2 CNV are shown. Dashed red lines indicated the CNV limits that define each CNV level at low RPP30 copies. The RPP30 copies for these 4 specimens ranged from 277 to 383 RPP30 copies/well.

### DETAILED DESCRIPTION

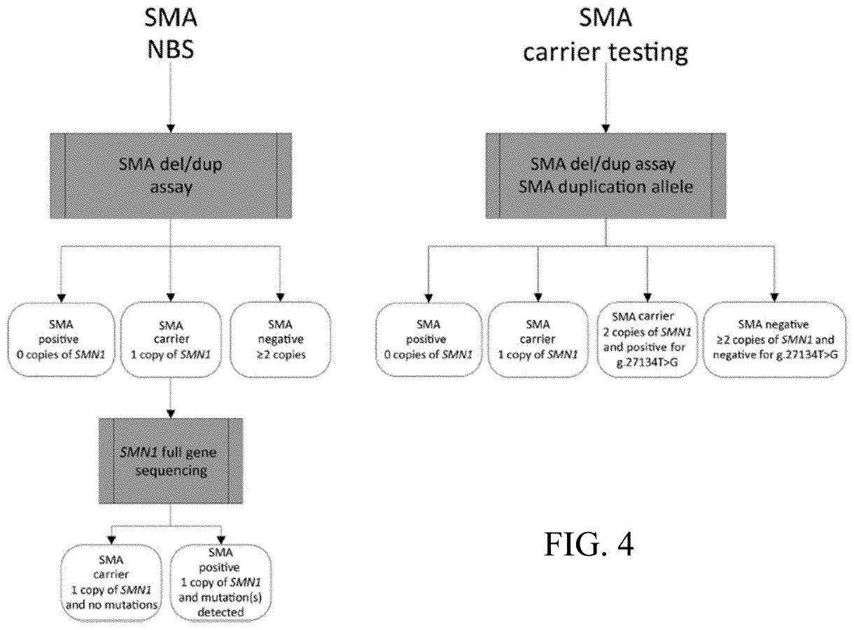

This document provides methods and materials for assessing and/or treating a mammal (e.g., a human) having, or at risk of developing, a spinal condition (e.g., SMA). In some cases, a mammal can be identified as having, or as being likely to develop, a spinal condition (e.g., SMA), using a sensitive and specific method (e.g., multiplex dPCR such as multiplex ddPCR). For example, a mammal (e.g., a human) can be identified as having or as being at risk of developing a spinal condition (e.g., SMA) based, at least in part, on the modification of nucleic acid (e.g., one or more genes) encoding a SMN polypeptide (e.g., a homozygous deletion of exon 7 in a genomic SMN1 nucleic acid encoding a SMN polypeptide and the genomic copy number of SMN2 nucleic acid in a sample from a mammal. For example, the presence of a homozygous deletion of exon 7 in a genomic SMN1 nucleic acid (e.g., homozygous deletion of exon 7 of the genomic SMN1 gene) encoding a SMN polypeptide can be used to identify a mammal as having, or as being at risk of developing, a spinal condition (e.g., SMA), and the genomic copy number of SMN2 nucleic acid (e.g., the genomic copy number of the SMN2 gene) encoding a SMN polypeptide) encoding a SMN polypeptide can be used to predict the severity of the SMA (e.g., fewer genomic copies such as 1 or 2 copies of SMN2 nucleic acid encoding a SMN polypeptide can indicate that the SMA is likely to be more severe, while more genomic copies such as 3, 4, or 5 copies of SMN2 nucleic acid encoding a SMN polypeptide can indicate that the SMA is likely to be less severe). In some cases, multiplex dPCR (e.g., multiplex ddPCR such as high-throughput multiplex ddPCR) can be used to simultaneously determine the presence of homozygous deletion of exon 7 in a genomic SMN1 nucleic acid encoding a SMN polypeptide and the genomic copy number of SMN2 nucleic acid encoding a SMN polypeptide in a sample from a mammal. An exemplary high-throughput multiplex ddPCR method that can be used in newborn screening to simultaneously determine the presence of homozygous deletion of exon 7 in a genomic SMN1 nucleic acid encoding a SMN polypeptide and the genomic copy number of SMN2 nucleic acid encoding a SMN polypeptide is shown in FIG. 4.

Any type of mammal can be assessed and/or treated as described herein. Examples of mammals that can be assessed and/or treated as described herein include, without limitation, primates (e.g., humans and monkeys), dogs, cats, horses, cows, pigs, sheep, rabbits, mice, and rats. In some cases, the mammal can be a human. A mammal can be any age. In some cases, a mammal can be a prenatal mammal (e.g., a human before birth). In some cases, a mammal can be a newborn mammal (e.g., a human from about birth to about 2 months of age). In some cases, a mammal can be an infant mammal (e.g., a human from about 2 months of age to about 1 year of age). In some cases, a mammal can be an adolescent mammal (e.g., a human from about 1 year of age to about 17 years of age). In some cases, a mammal can be an adult mammal (e.g., a human about 18 years of age or older). For example, a mammal having, or suspected of having, SMA can be assessed as described herein (e.g., for the presence of homozygous deletion of exon 7 in a genomic SMN1 nucleic acid encoding a SMN polypeptide and the genomic copy number of SMN2 nucleic acid encoding a SMN polypeptide in a sample from a mammal), and, optionally, can be treated as described herein.

When assessing and/or treating a mammal (e.g., a human) having, or at risk of developing, a spinal condition such as SMA, the SMA can be any type of SMA. Examples of types of SMA include, without limitation, Type 1 SMA (also called Werdnig-Hoffmann Disease), Type 2 SMA, Type 3 SMA (also called Kugelberg-Welander or Juvenile Spinal Muscular Atrophy), and Type 4 SMA. SMA can have any severity (e.g., severe, intermediate, or mild) of symptoms. In some cases, the type of SMA can be indicative of the severity of the SMA. For example, Type 1 SMA can be a severe SMA, Type 2 SMA can be an intermediate SMA, and Type 3 SMA can be a mild SMA.

When assessing a sample (e.g., a sample from a mammal) as described herein (e.g., for the presence of homozygous deletion of exon 7 in a genomic SMN1 nucleic acid encoding a SMN polypeptide and the genomic copy number of SMN2 nucleic acid encoding a SMN polypeptide in a sample from a mammal), the nucleic acid encoding a SMN polypeptide can encode any SMN polypeptide. Nucleic acid encoding a SMN polypeptide can encode a wild type SMN polypeptide or a mutant SMN polypeptide.

When assessing a sample (e.g., a sample from a mammal) as described herein (e.g., for the presence of homozygous deletion of exon 7 in a genomic SMN1 nucleic acid encoding a SMN polypeptide and the genomic copy number of SMN2 nucleic acid encoding a SMN polypeptide), the sample can be any appropriate sample from a mammal (e.g., a human). In some cases, a sample can be a biological sample. A biological sample can include nucleic acid (e.g., DNA such as genomic DNA). A biological sample can be a fresh sample or a fixed sample. A biological sample can be a processed (e.g., dried) sample such as a DBS. Examples of biological samples that can be assessed as described herein include, without limitation, fluid samples (e.g., blood, serum, plasma, urine, and saliva), cellular samples (e.g., buccal samples), and tissue samples (e.g., muscle tissue samples). In some cases, a biological sample can be a blood sample (e.g., a peripheral blood sample such as a whole blood sample or a DBS). In some cases, one or more molecules (e.g., DNA such as genomic DNA) can be isolated from a biological sample.

A mammal (e.g., a sample from a mammal) can be assessed as described herein (e.g., for the presence of homozygous deletion of exon 7 in a genomic SMN1 nucleic acid encoding a SMN polypeptide and the genomic copy number of SMN2 nucleic acid encoding a SMN polypeptide) using any appropriate dPCR method. In some cases, a dPCR method can be a ddPCR method. dPCR can include partitioning a single sample (e.g., a single DNA sample such a genomic DNA) into a plurality of partitions (e.g., droplets), performing PCR within each sample to amplify a target nucleic acid in the presence of fluorescently-labeled probes that can hybridize to amplified target nucleic acid where hybridization between a fluorescently-labeled probe and the amplified target nucleic acid emits a fluorescent signal, detecting the presence of the fluorescent signal within each droplet, and determining the presence and/or amount of target nucleic acid based on the presence, absence, or amount of the fluorescent signal.

In some cases, dPCR methods (e.g., ddPCR methods) provided herein can include PCR amplification of two or more (e.g., 2, 3, 4, or more) different target nucleic acids within a single sample (e.g., multiplexed PCR amplification). For example, ddPCR methods provided herein can be used to simultaneously detect the presence of homozygous deletion of exon 7 in a genomic SMN1 nucleic acid encoding a SMN polypeptide and the genomic copy number of SMN2 nucleic acid encoding a SMN polypeptide.

dPCR methods (e.g., ddPCR methods) provided herein can include any appropriate number of partitions. In some cases, dPCR methods can include from about 5,000 partitions to about 25,000 partitions. When a ddPCR method provided herein is a dPCR method, the ddPCR method can include any appropriate number of droplets. In some cases, ddPCR methods can include from about 5,000 droplets to about 25,000 droplets (e.g., from about 8,000 droplets to about 25,000 droplets, from about 10,000 droplets to about 25,000 droplets, from about 12,000 droplets to about 25,000 droplets, from about 15,000 droplets to about 25,000 droplets, from about 18,000 droplets to about 25,000 droplets, from about 20,000 droplets to about 25,000 droplets, from about 5,000 droplets to about 22,000 droplets, from about 5,000 droplets to about 20,000 droplets, from about 5,000 droplets to about 18,000 droplets, from about 5,000 droplets to about 15,000 droplets, from about 5,000 droplets to about 12,000 droplets, from about 7,000 droplets to about 23,000 droplets, from about 10,000 droplets to about 20,000 droplets, from about 12,000 droplets to about 18,000 droplets, from about 5,000 droplets to about 10,000 droplets, from about 10,000 droplets to about 15,000 droplets, or from about 15,000 droplets to about 20,000 droplets).

dPCR methods (e.g., ddPCR methods) provided herein can include partitions (e.g., droplets) of appropriate size. In some cases, when partitions are droplets, the droplets can be substantially the same size (e.g., are uniform-size droplets).

Partitions (e.g., droplets) in dPCR methods (e.g., ddPCR methods) provided herein can be generated using any appropriate method. In some cases, when partitions are droplets, the droplets can be generated using a water oil emulsion technique. When using a water oil emulsion technique to generate droplets for use in ddPCR, the droplets can include a sample (e.g., a nanoliter-size sample) encapsulated in oil. In some cases, droplets can be generated using a droplet generator (e.g., an automated droplet generator). In some cases, droplets can be generated as described in Example 1. In some cases, droplets can be generated as described elsewhere (see, e.g., Vidal-Folch et al., Clinical Chemistry 64:1753-1761 (2018)).

PCR amplification in dPCR methods (e.g., ddPCR methods) provided herein can include amplifying a target nucleic acid in the presence of fluorescently-labeled probes that can hybridize to amplified target nucleic acid where hybridization between a fluorescently-labeled probe and the amplified target nucleic acid emits a fluorescent signal. PCR amplification can include any appropriate PCR reaction mixture. A PCR reaction mixture can include nucleic acid template (e.g., DNA such as genomic DNA), at least one oligonucleotide primer pair, at least one probe that can hybridize to an amplicon generated by the oligonucleotide primer pair, nucleoside triphosphates containing deoxyribose (dNTPs), and a polymerase. For example, a PCR reaction mixture can include a first oligonucleotide primer pair for amplification of exon 7 of a SMN1 nucleic acid encoding a SMN polypeptide, a probe having a first fluorescent label that can hybridize to a SMN1 exon 7 amplicon generated by the first oligonucleotide primer pair, a second oligonucleotide primer pair for amplification of SMN2 nucleic acid encoding a SMN polypeptide, a probe having a second fluorescent label that can hybridize to a SMN2 amplicon generated by the second oligonucleotide primer pair, dNTPs, and a polymerase.

dPCR methods (e.g., ddPCR methods) provided herein can include any appropriate nucleic acid template. In some cases, a nucleic template can be DNA (e.g., genomic DNA). For example, a nucleic acid template can be genomic DNA isolated from a sample obtained from a mammal (e.g., a human). In some cases, a nucleic acid template can be diluted.

dPCR methods (e.g., ddPCR methods) provided herein can include any appropriate dNTPs. Examples of dNTPS include, without limitation, dATP, dTTP, dGTP, dCTP, and dUTP. In some cases, a PCR reaction mixture can include dATPs, dTTPs, dGTPs, and dCTPs. In some cases, a PCR reaction mixture does not include dUTP.

dPCR methods (e.g., ddPCR methods) provided herein can include any appropriate polymerase. A polymerase can be a DNA polymerase or a RNA polymerase. A polymerase can be a naturally occurring polymerase or a synthetic polymerase. A polymerase can be a heat-tolerant polymerase. A polymerase can be a heat-activated polymerase. A polymerase can be from any appropriate organism (e.g., a bacterium such as Thermus aquaticus). Examples of polymerases that can be used in a PCR reaction mixture of a PCR amplification in ddPCR methods provided herein include, without limitation, Taq polymerase.

dPCR methods (e.g., ddPCR methods) provided herein can include any appropriate oligonucleotides (e.g., oligonucleotide primers and oligonucleotide probes). In some cases, an oligonucleotide can be a primer (e.g., can serve as a starting point for synthesis of a target nucleic acid). For example, a primer can be (e.g., can be designed to be) complementary to a target nucleic acid such that when the primer is hybridized to the target nucleic acid a polymerase (e.g., a DNA polymerase or an RNA polymerase) can add one or more nucleotides the 3′-end of a primer to synthesize the target nucleic acid. In some cases, an oligonucleotide can be a probe (e.g., can be used to detect the presence or absence of a target nucleic acid). For example, a probe can be (e.g., can be designed to be) complementary to a target nucleic acid (e.g., an amplicon generated by an oligonucleotide primer pair) such that hybridization of the probe to its target nucleic acid can indicate the presence of the target nucleic acid. In some cases, ddPCR methods can include at least one assay mix that includes at least one oligonucleotide primer pair and at least one probe that can hybridize to an amplicon generated by the oligonucleotide primer pair.

Oligonucleotides (e.g., oligonucleotide primers and oligonucleotide probes) can include any appropriate type of nucleic acid. For example, an oligonucleotide can be DNA, RNA, or a combination thereof.

Oligonucleotides (e.g., oligonucleotide primers and oligonucleotide probes) can be single stranded nucleic acid or double stranded nucleic acid.

Oligonucleotides (e.g., oligonucleotide primers and oligonucleotide probes) can include any appropriate number of nucleotides. For example, an oligonucleotide can include from about 8 nucleotides to about 35 nucleotides (e.g., from about 10 nucleotides to about 35 nucleotides, from about 12 nucleotides to about 35 nucleotides, from about 15 nucleotides to about 35 nucleotides, from about 18 nucleotides to about 35 nucleotides, from about 20 nucleotides to about 35 nucleotides, from about 23 nucleotides to about 35 nucleotides, from about 25 nucleotides to about 35 nucleotides, from about 8 nucleotides to about 32 nucleotides, from about 8 nucleotides to about 30 nucleotides, from about 8 nucleotides to about 27 nucleotides, from about 8 nucleotides to about 25 nucleotides, from about 8 nucleotides to about 22 nucleotides, from about 8 nucleotides to about 20 nucleotides, from about 8 nucleotides to about 17 nucleotides, from about 10 nucleotides to about 30 nucleotides, from about 12 nucleotides to about 28 nucleotides, from about 15 nucleotides to about 25 nucleotides, from about 10 nucleotides to about 20 nucleotides, from about 15 nucleotides to about 25 nucleotides, or from about 20 nucleotides to about 30 nucleotides). In cases where an oligonucleotide is a primer, the oligonucleotide can include from about 15 to about 30 nucleotides. In cases where an oligonucleotide is a probe, the oligonucleotide can include from about 30 to about 200 nucleotides.

Oligonucleotides (e.g., oligonucleotide primers and oligonucleotide probes) can include one or more modified nucleotides. Examples of modified nucleotides include, without limitation, nucleotides in which the 5-carbon sugar (e.g., ribose or deoxyribose) in the nucleotide includes a bridge connecting the 2′ oxygen and 4′ carbon, isoguanine (iso-dG), and 5′-methylisocytosine (iso-dC). In some cases, an oligonucleotide provided herein can include one or more nucleotides in which a 5-carbon sugar in the nucleotide includes a bridge connecting the 2′ oxygen and 4′ carbon. When an oligonucleotide includes one or more nucleotides in which a 5-carbon sugar in the nucleotide includes a bridge connecting the 2′ oxygen and 4′ carbon, the oligonucleotide can be referred to as a locked nucleic acid (LNA) oligonucleotide.

In some cases, oligonucleotides (e.g., oligonucleotide primers and oligonucleotide probes) can include one or more labels. For example, an oligonucleotide can include one or more (e.g., 1, 2, 3, or more) labels. A label can be attached (e.g., covalently attached) to an oligonucleotide at any appropriate position. In some cases, a label can be internal (e.g., within the oligonucleotide). In some cases, a label can be on one or more ends of an oligonucleotide. For example, a label can be attached (e.g., covalently attached) to the 5′ end of an oligonucleotide, to the 3′ end, or to both the 5′ end and the 3′ end of an oligonucleotide. In some cases, a label can be a fluorescent dye. Examples of fluorescent dyes include, without limitation, FAM, HEX, ATTO 532, ATTO Rho101, Alexa Fluor, fluorescein (6-FAM), 6-carboxy-4′,5′-dichloro-2′,7′-dimethoxyfluorescein (JOE), tetrachlorofluorescein (TET), carboxy-X-rhodamine (ROX), 5- (and 6) carboxytetramethylrhodamine (TAMRA), TEX 615, TYE 563, TYE 665, TYE 705, Texas Red-X, 3-(5,6,4′,7′-tetrachloro-5′-methyl-3′,6′-dipivaloylfluorescein-2-yl)-propanamidohexyl-1-O-(2-cyanoethyl)-(N,N-diisopropyl)-phosphoramidite (Yakima Yellow), Cyanine 3, Cyanine 5, and Cyanine 5.5, 2′-chloro-7′phenyl-1,4-dichloro-6-carboxy-fluorescein (VIC). In some cases, a label can be a fluorescent quencher. Examples of fluorescent quenchers include, without limitation, tetramethyl-rhodamine (TAM), ZEN™ quenchers, and dark quenchers (e.g., Iowa Black® fluorescent quenchers (IBFQ or IABkFQ)). In cases where an oligonucleotide is a probe, the probe can include a FAM at its 5′ and, an internal ZEN™, and a IBFQ at its 3′ end.

dPCR methods (e.g., ddPCR methods) can include any appropriate amount of oligonucleotides (e.g., oligonucleotide primers and oligonucleotide probes). For example, ddPCR methods can include from about 200 nM to about 1000 nM of oligonucleotides (e.g., in a PCR reaction mixture). In cases where an oligonucleotide is a primer, the oligonucleotide can include about 900 nM of oligonucleotide primers. In cases where an oligonucleotide is a probe, the oligonucleotide can include about 250 nM of oligonucleotide probes.

Oligonucleotides (e.g., oligonucleotide primers and oligonucleotide probes) can target (e.g., can be designed to target) any appropriate nucleic acid encoding a SMN polypeptide. In some cases, oligonucleotide primers can target exon 7 of SMN1 nucleic acid encoding a SMN polypeptide and oligonucleotide probes can target a SMN1 amplicon generated by (e.g., amplified by) the oligonucleotide primer pair. For example, PCR amplification including oligonucleotide primers that can target exon 7 of SMN1 nucleic acid encoding a SMN polypeptide and oligonucleotide probes that can target a SMN1 amplicon generated by the oligonucleotide primer pair can be performed under conditions that allow, when one or more SMN1 amplicons are generated by the oligonucleotide primer pair, the oligonucleotide probes to hybridize to one or more SMN1 amplicons. Exemplary oligonucleotide primers that can target exon 7 of SMN1 nucleic acid encoding a SMN polypeptide include, without limitation, oligonucleotide primers including the nucleotide sequence TCCATATAAAGCTATCTATATATAGCTATCTATG (SEQ ID NO:1), oligonucleotide primers including the nucleotide sequence TGTGAGCACCTTCCTTCTT (SEQ ID NO:2), oligonucleotide primers including the nucleotide sequence oligonucleotide primers including the nucleotide sequence TCCTTTATTTTCCTTACAGGGTTTC (SEQ ID NO:10), oligonucleotide primers including the nucleotide sequence ATTGTTTTACATTAACCTTTCAACTTTTT (SEQ ID NO:11), TCCTTTATTTTCCTTACAGGGTTTC (SEQ ID NO:13), and oligonucleotide primers including the nucleotide sequence ATTGTTTTACATTAACCTTTCAACTTTTT (SEQ ID NO:14). In some cases, an oligonucleotide primer pair that can target exon 7 of SMN1 nucleic acid encoding a SMN polypeptide can include a first oligonucleotide primer including the nucleotide sequence set forth in SEQ ID NO:1, SEQ ID NO:10 or SEQ ID NO:13 and a second oligonucleotide primer including the nucleotide sequence set forth in SEQ ID NO:2, SEQ ID NO:11, or SEQ ID NO:14. Exemplary oligonucleotide probes that can target a SMN1 amplicon generated by the oligonucleotide primer pair include, without limitation, oligonucleotide probes including the nucleotide sequence TCTGAAACCC (SEQ ID NO:3), oligonucleotide probes including the nucleotide sequence TTGAACATTTAAAAAGTT (SEQ ID NO:12), and oligonucleotide probes including the nucleotide sequence TGAACAGTTAAAAAGT (SEQ ID NO:15). In some cases, an oligonucleotide probe that can target a SMN1 amplicon generated by the oligonucleotide primer pair can include a fluorescent label and one or more fluorescent quenchers. For example, oligonucleotide primers that can target exon 7 of SMN1 nucleic acid encoding a SMN polypeptide and oligonucleotide probes can target a SMN1 amplicon generated by (e.g., amplified by) the oligonucleotide primer pair can be as set forth in Table 2.

In some cases, oligonucleotide primers can target SMN2 nucleic acid encoding a SMN polypeptide and oligonucleotide probes can target an SMN2 amplicon generated by (e.g., amplified by) the oligonucleotide primer pair. For example, PCR amplification including oligonucleotide primers that can target SMN2 nucleic acid encoding a SMN polypeptide and oligonucleotide probes can target an SMN2 amplicon generated by the oligonucleotide primer pair can be performed under conditions that allow, when one or more SMN2 amplicons are generated by the oligonucleotide primer pair, the oligonucleotide probes to hybridize to one or more SMN2 amplicons. Exemplary oligonucleotide primers that can target SMN2 nucleic acid encoding a SMN polypeptide include, without limitation, oligonucleotide primers including the nucleotide sequence TCCATATAAAGCTATCTATATATAGCTATCTATA (SEQ ID NO:4), and oligonucleotide primers including the nucleotide sequence TGTGAGCACCTTCCTTCTT (SEQ ID NO:5). In some cases, an oligonucleotide primer pair that can target SMN2 nucleic acid encoding a SMN polypeptide can include a first oligonucleotide primer including the nucleotide sequence set forth in SEQ ID NO:4 and a second oligonucleotide primer including the nucleotide sequence set forth in SEQ ID NO:5. Exemplary oligonucleotide probes that can target an SMN2 amplicon generated by the oligonucleotide primer pair include, without limitation, oligonucleotide probes including the nucleotide sequence TGTCTAAAACCCT (SEQ ID NO:6), and oligonucleotide probes including the nucleotide sequence GGGTTTTAGACA (SEQ ID NO:16). In some cases, an oligonucleotide probe that can target an SMN2 amplicon generated by the oligonucleotide primer pair can include a fluorescent label and one or more fluorescent quenchers. For example, oligonucleotide primers that can target SMN2 nucleic acid encoding a SMN polypeptide and oligonucleotide probes can target an SMN2 amplicon generated by (e.g., amplified by) the oligonucleotide primer pair can be as set forth in Table 2.

In some cases, oligonucleotide primers can target nucleic acid encoding a reference polypeptide and oligonucleotide probes can target a reference amplicon generated by (e.g., amplified by) the oligonucleotide primer pair. In some cases, nucleic acid encoding a reference polypeptide can be a conserved genomic nucleic acid encoding a conserved reference polypeptide. Examples of reference polypeptides that can be encoded by a conserved nucleic acid encoding a reference polypeptide include, without limitation, ribonuclease P (RPP30) and β-actin (ACTB) polypeptides. For example, PCR amplification including oligonucleotide primers that can target nucleic acid encoding a reference polypeptide and oligonucleotide probes that can target a reference amplicon generated by the oligonucleotide primer pair can be performed under conditions that allow, when one or more reference amplicons are generated by the oligonucleotide primer pair, the oligonucleotide probes to hybridize to one or more reference amplicons. Exemplary oligonucleotide primers that can target nucleic acid encoding a RPP30 polypeptide include, without limitation, oligonucleotide primers including the nucleotide sequence AGATTTGGACCTGCGAGCG (SEQ ID NO:7), oligonucleotide primers including the nucleotide sequence GAGCGGCTGTCTCCACAAGT (SEQ ID NO:8). In some cases, an oligonucleotide primer pair that can target nucleic acid encoding a RPP30 polypeptide can include a first oligonucleotide primer including the nucleotide sequence set forth in SEQ ID NO:7 and a second oligonucleotide primer including the nucleotide sequence set forth in SEQ ID NO:8. Exemplary oligonucleotide probes that can target a RPP30 amplicon generated by the oligonucleotide primer pair include, without limitation, oligonucleotide primers including the nucleotide sequence TTCTGACCTGAAGGCTCTGCGCG (SEQ ID NO:9). In some cases, an oligonucleotide probe that can target a RPP30 amplicon generated by the oligonucleotide primer pair can include a fluorescent label and one or more fluorescent quenchers. For example, oligonucleotide primers that can target SMN2 nucleic acid encoding a SMN polypeptide and oligonucleotide probes that can target nucleic acid encoding a reference polypeptide and oligonucleotide probes can target a reference amplicon generated by (e.g., amplified by) the oligonucleotide primer pair can be as set forth in Table 2.

dPCR methods (e.g., ddPCR methods) provided herein can include any appropriate PCR amplification. PCR amplification can include a denaturing phase, an annealing phase, and an extension phase. Each phase of an amplification cycle can include any appropriate conditions. In some cases, a denaturing phase can include a temperature of about 94° C. to about 98° C., and a time of about 20 seconds to about 60 seconds. For example, a denaturing phase can include a temperature of about 94° C. for about 30 seconds. In some cases, an annealing phase can include a temperature of about 50° C. to about 65° C., and a time of about 20 seconds to about 40 seconds. In some cases, an extension phase can include a temperature of about 55° C. to about 80° C., and a time of about 15 seconds per kb of the amplicon to be generated to about 30 seconds per kb of the amplicon to be generated. In some cases, annealing and extension phases can be performed in a single cycle. For example, an annealing and phase extension phase can include a temperature of about 58° C. for about 1 minute.

dPCR methods (e.g., ddPCR methods) provided herein can include any appropriate number of PCR amplification cycles. In some cases, PCR amplification can include from about 25 to about 50 cycles. For example, PCR amplification can include about 40 amplification cycles.

In some cases, when PCR conditions include a heat-activated polymerase, PCR amplification also can include an initialization step. For example, PCR amplification can include an initialization step prior to performing the PCR amplification cycles. In some cases, an initialization step can include a temperature of about 94° C. to about 98° C., and a time of about 1 minutes to about 10 minutes.

In some cases, PCR amplification also can include a final extension step. For example, PCR amplification can include a final extension step after performing the PCR amplification cycles. In some cases, a final extension step can include a temperature of about 70° C. to about 98° C., and a time of about 5 minutes to about 15 minutes. For example, a final extension step can include a temperature of about 98° C., and a time of about 10 minutes.

In some cases, PCR amplification also can include a hold step. For example, PCR amplification can include a hold step after performing the PCR amplification cycles, an optionally after performing any final extension step. In some case, a hold step can include a temperature of about 4° C. to about 15° C., for an indefinite amount of time.

dPCR methods (e.g., ddPCR methods) provided herein can include determining the presence, absence, or amount of target nucleic acid. In some cases, the presence, absence, or amount of target nucleic acid can be determined based on the presence or absence of hybridization between one or more oligonucleotide probes that can target an amplicon generated by an oligonucleotide primer pair and one or more amplicons. For example, detecting the presence or absence of hybridization between one or more oligonucleotide probes that can target an amplicon generated by an oligonucleotide primer pair and one or more amplicons can include detecting the presence, absence, or amount of a fluorescent signal emitted when hybridization between one or more oligonucleotide probes that can target an amplicon generated by an oligonucleotide primer pair and one or more amplicons occurs.

In some cases, dPCR methods (e.g., ddPCR methods) provided herein can include PCR amplification of two or more (e.g., 2, 3, 4, or more) different target nucleic acids within a single sample (e.g., multiplexed PCR amplification). For example, fluorescently-labeled probes that can hybridize to an amplicon generated by a first oligonucleotide primer pair that can amplify a first target nucleic acid can include a first fluorescent label, fluorescently-labeled probes that can hybridize to an amplicon generated by a second oligonucleotide primer pair that can amplify a second target nucleic acid can include a second fluorescent label, and so on. For example, when assessing a sample (e.g., a sample from a mammal) for the presence of homozygous deletion of exon 7 in a genomic SMN1 nucleic acid encoding a SMN polypeptide and the genomic copy number of SMN2 nucleic acid encoding a SMN polypeptide, ddPCR can include performing PCR amplification using (a) a first oligonucleotide primer pair that can amplify exon 7 of SMN1 nucleic acid encoding a SMN polypeptide in the presence of fluorescently-labeled probes including a first fluorescent label that can hybridize to a SMN1 amplicon generated by the first oligonucleotide primer pair such that hybridization between the fluorescently-labeled probe and the SMN1 amplicon emits a first fluorescent signal, and (b) a second oligonucleotide primer pair that can amplify SMN2 nucleic acid encoding a SMN polypeptide in the presence of fluorescently-labeled probes including a second fluorescent label that can hybridize to a SMN2 amplicon generated by the second oligonucleotide primer pair such that hybridization between the fluorescently-labeled probe and the SMN1 amplicon emits a second fluorescent signal. In some cases, the first and second fluorescent signals do not have overlapping wavelengths. For example, fluorescently-labeled probes (e.g., fluorescently-labeled probes that can hybridize to a SMN1 amplicon generated by a first oligonucleotide primer pair) having a first fluorescent label can include a FAM fluorescent label and fluorescently-labeled probes (e.g., fluorescently-labeled probes that can hybridize to a SMN2 amplicon generated by a second oligonucleotide primer pair) having a second fluorescent label can include a HEX fluorescent label.

dPCR methods (e.g., ddPCR methods) provided herein can include detecting one or more fluorescent signals emitted when hybridization between one or more oligonucleotide probes that can target an amplicon generated by an oligonucleotide primer pair and one or more amplicons occurs. In cases where two or more (e.g., 2, 3, 4, or more) fluorescent signals are emitted, the two or more fluorescent signals can be detected simultaneously. In some cases, ddPCR methods can include negative droplets (e.g., droplets that emit no fluorescent signal (e.g., no detectable fluorescent signal). For example, when PCR amplification within a droplet produces no detectable amplicons, no hybridization of a fluorescently-labelled probe and an amplicon can occur, and no fluorescent signal is emitted.

Fluorescent signals in dPCR methods (e.g., ddPCR methods) provided herein can be detected using any appropriate method. In some cases, a fluorescent signal can be measured as signal amplitude. In some cases, fluorescent signals can be detected using a fluorescent reader (e.g., a QX200 Droplet Reader). In some cases, fluorescent signals can be detected as described in Example 1.

In some cases, dPCR methods (e.g., ddPCR methods) provided herein can be performed without (e.g., do not require) standard curves. For example, ddPCR methods provided herein can include a Poisson calculation across all droplets. A Poisson calculation across all droplets can allow a concentration determination (e.g., an absolute concentration determination) for a target nucleic acid. For example, a Poisson calculation across all droplets can eliminate the need for standard curves (e.g., standard curves of reference standards) and/or endogenous controls.

In some cases, dPCR methods (e.g., ddPCR methods) provided herein can be performed without (e.g., do not require) repeat analyses. For example, the presence of homozygous deletion of exon 7 in a genomic SMN1 nucleic acid encoding a SMN polypeptide and the genomic copy number of SMN2 nucleic acid encoding a SMN polypeptide in a sample from a mammal can be determined using a single assay of ddPCR methods provided herein.

In some cases, methods described herein also can include covariates and/or additional newborn screening marker results. Examples of covariates that can be included in methods descried herein include, without limitation, gender, birthweight, and gestational age.

In some cases, methods described herein also can include confirming diagnosis with one or more additional tests used to diagnose SMA. Examples of tests used to diagnose SMA include, without limitation, physical examinations (e.g., physical examinations that can test gross motor abilities), electromyography (EMG) (e.g., to measure a muscle's ability to respond to electrical stimulation), and muscle biopsy tests (e.g., to examine muscle for signs of muscle degeneration).

dPCR methods (e.g., ddPCR methods) provided herein can be used to identify a mammal as having, or as being at risk of developing, a spinal condition (e.g., SMA) based, at least in part, on the presence or absence of homozygous deletion of exon 7 in a genomic SMN1 nucleic acid encoding a SMN polypeptide and the genomic copy number of SMN2 nucleic acid encoding a SMN polypeptide. For example, when the presence of homozygous deletion of exon 7 in a genomic SMN1 nucleic acid encoding a SMN polypeptide and the genomic copy number of SMN2 nucleic acid encoding a SMN polypeptide are identified in a sample from a mammal, the mammal can be identified as having, or as being at risk of developing, a spinal condition (e.g., SMA). In addition, the genomic copy number of SMN2 nucleic acid encoding a SMN polypeptide also can be used to determine the severity of SMA. For example, two or fewer (e.g., 1 or 2) genomic copies of SMN2 nucleic acid encoding a SMN polypeptide can be associated with a more severe SMA phenotype. For example, three or more (e.g., 3, 4, 5, or more) genomic copies of SMN2 nucleic acid encoding a SMN polypeptide can be associated with a milder SMA phenotype.

In some cases, dPCR methods (e.g., ddPCR methods) provided herein can be used to identify a mammal as being a carrier of a spinal condition (e.g., SMA). For example, a mammal can be identified as being a carrier of a spinal condition based, at least in part, on the presence or absence of heterozygous deletion of exon 7 in a genomic SMN1 nucleic acid encoding a SMN polypeptide. For example, when the absence of a deletion of exon 7 in a first copy of a genomic SMN1 nucleic acid encoding a SMN polypeptide and the presence of a deletion of exon 7 in a second copy of a genomic SMN1 nucleic acid encoding a SMN polypeptide are identified in a sample from a mammal, the mammal can be identified as being a carrier of a spinal condition (e.g., SMA).

In some cases, after a mammal is identified as having, or as being at risk of developing, a spinal condition (e.g., SMA) based, at least in part, on the presence or absence of homozygous deletion of exon 7 in a genomic SMN1 nucleic acid encoding a SMN polypeptide and the genomic copy number of SMN2 nucleic acid encoding a SMN polypeptide, a treatment can be selected for the mammal, and, optionally, can administered to the mammal. In cases where a mammal (e.g., a human) is identified as having 1 or 2 genomic copies of SMN2 nucleic acid encoding a SMN polypeptide, the mammal can be selected for treatment such as one or more appropriate SMA treatments. In cases where a mammal (e.g., a human) is identified as having 3 genomic copies of SMN2 nucleic acid encoding a SMN polypeptide, the mammal can be selected for monitoring and/or can be selected for treatment (e.g., treatment at a later date). In cases where a mammal (e.g., a human) is identified as having 4 or 5 genomic copies of SMN2 nucleic acid encoding a SMN polypeptide, the mammal can be selected for monitoring and/or can be selected as not needing treatment for SMA.

When methods described herein include selecting a mammal identified as having, or as being at risk of developing, a spinal condition (e.g., SMA), for monitoring, the monitoring can include any one or more additional tests used to diagnose SMA. Examples of tests used to diagnose SMA include, without limitation, physical examinations (e.g., physical examinations that can test gross motor abilities), EMG (e.g., to measure a muscle's ability to respond to electrical stimulation), and muscle biopsy tests (e.g., to examine muscle for signs of muscle degeneration).

When methods described herein include selecting, and, optionally, treating a mammal identified as having, or as being at risk of developing, SMA, the treatment can include any one or more appropriate SMA treatments. In some cases, selecting a treatment for a mammal identified as having, or as being at risk of developing, SMA, the selection can include selection of one or more appropriate SMA treatments, selection of dosage amounts of one or more appropriate SMA treatments, and/or selection of administration frequency of one or more appropriate SMA treatments. In some cases, a SMA treatment can reduce or eliminate one or more symptoms of SMA. Examples of symptoms of SMA include, without limitation, delays in development of gross motor skills (e.g., sitting or walking), loss of gross motor skills already mastered, breathing problems, trouble feeding, limp or floppy muscles, poor head control, muscle weakness that grows worse, frequent respiratory infections (e.g., frequent respiratory infections that get worse with each infection), nasal-sounding speech, poor posture (e.g., poor posture that gets worse over time), and scoliosis (e.g., scoliosis that gets worse over time). Examples of SMA treatments include, without limitation, administration of one or more agents that can increase exon 7 inclusion in SMN1 messenger ribonucleic acid (mRNA) such as nusinersen (SPINRAZA®), administration of one or more agents that can provide SMN polypeptide function, administration of one or more agents that can replace and/or correct of the deletion of exon 7 in genomic SMN1 nucleic acid that can encode a SMN polypeptide, administration of one or more agents that can protect motor neurons affected by SMA, administration of one or more agents that can prevent and/or restore the loss of muscle function associated with SMA, occupational therapy, physical therapy, and speech and language therapy.

In some cases, a treatment can be selected, and, optionally, administered, to a mammal identified as having, or as being at risk of developing, a spinal condition (e.g., SMA) based, at least in part, on the presence of homozygous deletion of exon 7 in a genomic SMN1 nucleic acid encoding a SMN polypeptide and/or the genomic copy number of SMN2 nucleic acid encoding a SMN polypeptide, as described elsewhere (see, e.g., Glascock et al., Journal of Neuromuscular Diseases 5:145-158 (2018)).

In cases where a mammal identified as having, or as being at risk of developing, a spinal condition (e.g., SMA) based, at least in part, on the presence or absence of homozygous deletion of exon 7 in a genomic SMN1 nucleic acid encoding a SMN polypeptide and the genomic copy number of SMN2 nucleic acid encoding a SMN polypeptide, is treated by administering to the mammal one or more agents that can replace and/or correct of the deletion of exon 7 in genomic SMN1 nucleic acid that can encode a SMN polypeptide, methods described herein can be used to monitor the efficacy of treatment. For example, the methods described herein can be used to determine whether or not the deletion of exon 7 in genomic SMN1 nucleic acid that can encode a SMN polypeptide has been altered. For example, the methods described herein can be used to determine a copy number of SMN1 nucleic acid that can encode a SMN polypeptide.

Also provided herein are kits that can include oligonucleotides provided herein (e.g., oligonucleotide primers and oligonucleotide probes can target any appropriate nucleic acid encoding a SMN polypeptide). For example, a kit provided herein can include a first oligonucleotide primer pair for amplification of exon 7 of a SMN1 nucleic acid encoding a SMN polypeptide, where the first oligonucleotide primer pair can be used to generate a SMN1 exon 7 amplicon, and a first probe having a first fluorescent label that can hybridize to that SMN1 exon 7 amplicon, and can include a second oligonucleotide primer pair for amplification of SMN2 nucleic acid encoding a SMN polypeptide, where the second oligonucleotide primer pair can be used to generate a SMN2 amplicon, and a second probe having a second fluorescent label that can hybridize to that SMN2 amplicon. In some cases, the first fluorescent label and the second fluorescent label can be different. In some cases, the fluorescent probe also can include one or more fluorescent quenchers (e.g., an internal quencher and a terminal quencher). In some cases, the probes included in a kit provided herein can be LNA oligonucleotide probes. For example, a kit can include one or more oligonucleotide primer pairs and one or more probes as set forth in Table 2. In some cases, a kit provided herein also can include packaging and/or a label that indicates how the contents are to be used (e.g., to assessing a sample for the presence of homozygous deletion of exon 7 in a genomic SMN1 nucleic acid encoding a SMN polypeptide and the genomic copy number of SMN2 nucleic acid encoding a SMN polypeptide as described herein).

The invention will be further described in the following examples, which do not limit the scope of the invention described in the claims.

### EXAMPLES

### Example 1: Newborn Screening for Spinal Muscular Atrophy by Droplet Digital PCR

### Materials and Methods

### Clinical Specimens

Residual blood, chorionic villus, and cultured cells were utilized with Mayo Clinic IRB approval. Anonymized infant DBS were collected on Whatman 903 Protein Saver Cards (GE Healthcare, Pittsburgh, Pa.). Otherwise, DBS were prepared from anonymized blood samples. Donor demographic data are provided in Table 1.

TABLE 1
Study Sample Demographics.

| | | | Gestational | Birth |
|---|---|---|---|---|
| | | Age at | Age | Weight |
| Cohort | Sex | Collection | (weeks) | (g) |
| Infants ≤ 7 days | 677 Females | 0-7 days | 23.4-42.1 | 600-6290 |
| (N = 1388) | 711 Males | (1.2 days) | (39.1) | (3400) |
| Infants > 7 days | 73 Females | 8 days-72.8 years | 24.3-38 | 680-4387 |
| and Adults | 69 Males | (28 days) | (33.4) | (1630) |
| (N = 142) | | | | |
| SMA positives | 6 Females | 12 days-38 years | N/A | N/A |
| (N = 12) | 6 Males | (77 days) | | |

Median values are shown in parentheses.
N/A, Not available

### DNA Extraction

The DBS DNA extraction protocol was performed as described elsewhere (see, e.g., Vidal-Folch et al., J Mol Diagn, 19:755-65 (2017)). Blood specimens were extracted by either Autopure (AutoGen, Holliston, Mass.) or MagNA Pure (Roche Diagnostics, Indianapolis, Ind.) methods. Cultured cells from amniotic fluid, chorionic villus or fibroblasts were extracted with Autopure. These extraction processes were verified for successful ddPCR performance.

### Droplet Digital PCR

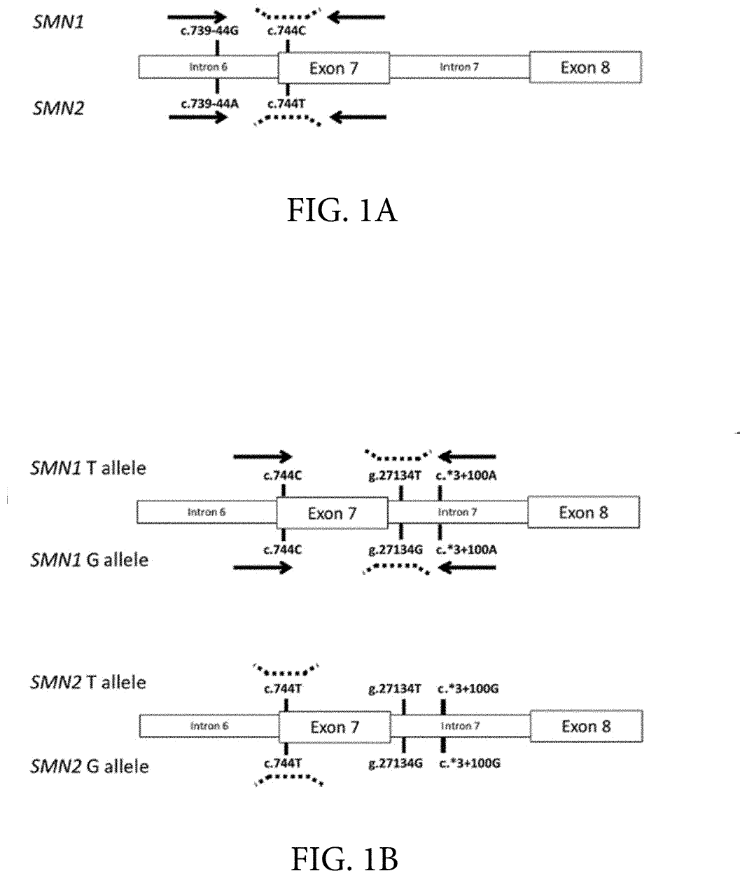

DdPCR primers and probes (FIGS. 1A and 1B, Table 2) were custom synthesized (IDT, Coralville, Iowa, USA). DdPCR reactions were assembled by a liquid-handling system in 96-well PCR plates. For the SMA CNV assay, reactions contained 11 μL of 2×ddPCR Supermix (no dUTP) (Bio-Rad, Pleasanton, Calif., USA), 1.1 μL of SMN1 CNV mix, 1.1 μL of SMN2 CNV mix, 1.1 μL of RPP30 CNV mix, 1.1 μL of Hind III, 4.4 μL of DNA and 2.2 μL of water. For the SMA [2+0] assay, reactions contained 11 μL of 2×ddPCR Supermix (no dUTP) (Bio-Rad, Pleasanton, Calif., USA), 1.1 μL of SMN1 Wild-Type (WT) mix, 1.1 μL of SMN1 Mutant (MUT) mix, 1.1 μL of SMN2 dark probe (500 nM), 1.1 μL of Hind III, 4.4 μL of DNA and 2.2 μL of water. The final concentrations of primers and probes were 900 nM and 250 nM, respectively. For carrier testing, two wells per sample were used, one for SMA CNV and one for SMA [2+0]. Multiwell plates were sealed at 180° C. for 4 seconds with foil (Bio-Rad, Pleasanton, Calif., USA), vortexed briefly, centrifuged, and placed in the droplet generator (AutoDG, Bio-Rad, Pleasanton, Calif., USA) by loading 20 μL it mix and 70 μL droplet oil into a DG-32 cartridge (Bio-Rad, Pleasanton, Calif., USA). After droplet generation, plates were resealed. Endpoint PCR conditions: 95° C. for 10 minutes; 40 cycles of denaturation at 94° C. for 30 seconds; annealing and extension at 58° C. for 1 minute; and a final step at 98° C. for 10 minutes with a thermal cycler (Applied Biosystems, Foster City, Calif., USA) enabled amplification. Plates were stored at 4° C. until read on a QX200 Droplet Reader (Bio-Rad, Pleasanton, Calif., USA).

TABLE 2
Primer and Probe Sequences for ddPCR Assays.

| | | | SEQ | Probe | | SEQ |
|---|---|---|---|---|---|---|
| Assay | Primer Name | Primer Sequence | ID NO: | Name | Probe Sequence | ID NO: |
| SMN1 | SMN1 | TCCATATAAAGCTATCTATATATAGCTAT | 1 | SMN1 | FAM/TC + T + G + AA + A + C + | 3 |
| CMV | forward | CTATG | | FAM | CC/3IABkFQ | |
| | | | | probe | | |
| Assay | SMN1/2 | TGTGAGCACCTTCCTTCTT | 2 | SMN1 | HEX/TC + T + G + AA + A + C + | 3 |
| Mix | reverse | | | HEX | CC/3IABkFQ | |
| | | | | probe | | |
| SMN2 | SMN2 | TCCATATAAAGCTATCTATATATAGCTAT | 4 | SMN2 | FAM/TG + TC + A + AAAC + C + | 6 |
| CMV | forward | CTATA | | FAM | CT/3IABkFQ | |
| | | | | probe | | |
| Assay | SMN1/2 | TGTGAGCACCTTCCTTCTT | 5 | | | |
| Mix | reverse | | | | | |
| RPP30 | RPP30 | AGATTTGGACCTGCGAGCG | 7 | RPP30 | HEX-TTCTGACCTGAAGGCTCTGCGCG- | 9 |
| CNV | forward | | | probe | ZEN/IBFQ | |
| Assay | RPP30 | GAGCGGCTGTCTCCACAAGT | 8 | | | |
| Mix | reverse | | | | | |
| SMN1 | SMN1 | TCCTTTATTTTCCTTACAGGGTTTC | 10 | SMN | HEX/TT + GAA + C + A + T + | 12 |
| WT | Forward | | | WT | TTA + AAA + A + G + | |
| | | | | probe | TT/IABkFQ | |
| Assay | SMN1 | ATTGTTTTACATTAACCTTTCAACTTTTT | 11 | | | |
| mix | Reverse | | | | | |
| SMN1 | SMN1 | TCCTTTATTTTCCTTACAGGGTTTC | 13 | SMN | FAM/TG + AA + C + A + G + | 15 |
| MUT | Forward | | | MUT | TTAA + A + A + AGT/3IABkFQ | |
| | | | | probe | | |
| Assay | SMN1 | ATTGTTTTACATTAACCTTTCAACTTTTT | 14 | | | |
| Mix | Reverse | | | | | |
| SMN2 | | | | SMN2 | G + G + GTT + T + T + AG + | 16 |
| Dark | | | | dark | ACA/3IABkFQ | |
| Probe | | | | probe | | |

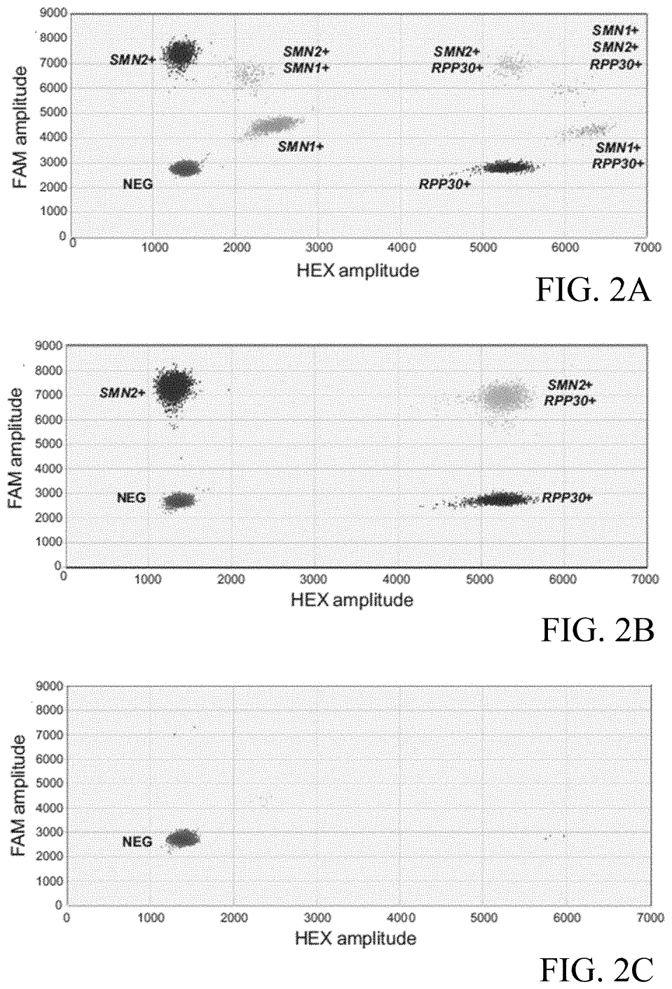

Bases in bold font indicate nucleotides which are unique to SMN1 or SMN2.
**SMN1 probe mix for the SMA ddPCR assay was prepared with 50% FAM probe and 50% of HEX probe. This probe mixture
positions the SMN1 droplet cluster at the center of the 2D fluroescent plot (yellow cluster in FIG. 2A), where it
can be clearly distinguished from SMN2 droplet clusters.

### SMN1 [2+0] Allele

SMN1 [2+0] assay was completed with primers for SMN1 exon 7 c.744C and c.*3+100A>G (NM_022874; FIG. 1B and Table 2). Probes for SMN1 g.27134T>G, a haplotype indicative of a [2+0] allele, as described elsewhere were used (see, e.g., Luo et al., Genet Med, 16:149-56 (2014)). An SMN2 dark probe to the c.744T nucleotide blocked SMN2 product formation (FIG. 1B, Table 2).

### SMN1-Specific Sanger Sequencing

TaKaRa LA Taq DNA Polymerase Hot-Start Version (Clontech Laboratories, Mountain View, Calif., USA) amplified exons 2-7 of SMN1 in 50 μL of 5 μL of 10×LA PCR Buffer II, 400 mM of each dNTP, 0.4 μM of each primer (Table 3), 2.5 units of LA Taq enzyme with 1M betaine. Target amplification with a thermal cycler (Thermo Fischer Scientific, Waltham, Mass., USA) at 94° C., 30 cycles of 10 seconds at 98° C., 15 minutes at 62° C., increasing to 20 seconds/cycle after 10 cycles, and a final step of 15 minutes at 72° C. A 13.4 kb long-range product was visualized on a 0.7% agarose gel with 1×TAE. SMN1 specificity was confirmed by the absence of three SMN2 sequence variants. Nested PCR amplification of SMN1 exons 2 through 7 used the KAPA2G Robust HotStart Kit (KAPA Biosystems, Wilmington, Mass.) in 10 μL with 2 μL KAPA2G buffer A, 0.2 mM dNTP mix, 0.4 μM of each UPS-tagged primers (Table 3), 0.5 U KAPA2G polymerase, and 1 μL of long-range amplicon.

TABLE 3
SMN1 Sequencing Primers.

| Primer Name | Sequence | SEQ ID NO: | Amplicon size |
|---|---|---|---|
| SMN1_LR_F | TGTGTGGATTAAGATGACTC | 17 | 13.4 kb |
| SMN1_LR_R | GAAAGTATGTTTCTTCCACAT | 18 | |
| SMN1_UPS_1F | AGAAGTTACTACAAGCGGTCCT | 19 | 319 bp |
| SMN1_UPS_1R | CACAACTCCAGTGAGCGGAT | 20 | |
| SMN1_UPS_2F | GGATTAAGATGACTCTTGGTAC | 21 | 313 bp |
| SMN1_UPS_2R | GGAGGATATCACCTGATTTAACT | 22 | |
| SMN1_UPS_3F | GAGCCTTGAGACTAGCTT | 23 | 334 bp |
| SMN1_UPS_3R | AAGTATATGTCAATAGAAACACTG | 24 | |
| SMN1_UPS_4.4F | CTCCCCACTGATCAAAACGAG | 25 | 302 bp |
| SMN1_UPS_4.4R | TTTTTTTTGTATCCTTACCTCTT | 26 | |
| SMN1_UPS_4.5F | AGATGATAGTTTGCCCTCTTC | 27 | 281 bp |
| SMN1_UPS_4.5R | TTTTTTGTATCCTTACCTCTT | 28 | |
| SMN1_UPS_5F | TTCAATTTCTGGAAGCAGAGA | 29 | 383 bp |
| SMN1_UPS_5R | CAAAAGTTTCATGGGAGAGC | 30 | |
| SMN1_UPS_6F | ATTTATTAGGTAATCACCACTCT | 31 | 480 bp |
| SMN1_UPS_6R | TTCTACCCATTAGAATCTGGC | 32 | |
| SMN1_UPS_7F | TGGGCAACATAGCAAGACCTC | 33 | 439 bp |
| SMN1_UPS_7R | ACAATGCAAGAGTAATTTAAGCCT | 34 | |
| SMN1_UPS_8F | AGACTATCAACTTAATTTCTGATCA | 35 | 413 bp |
| SMN1_UPS_8R | AAGTATGAGAATTCTAGTAGGGA | 36 | |

Amplification and sequencing of exon 1 was performed separately. Exon amplification was achieved with KAPA2G Robust HotStart Kit in a 10 μL using 2 KAPA2G buffer A, 0.2 mM of each dNTP, 0.4 μM of each primer (Table 3), 0.5 U of KAPA2G polymerase, and 10-250 ng of genomic DNA. As SMN1 and SMN2 exon 1 are 100% identical, SMN1- and SMN2-specific alterations in exon 1 were indistinguishable.

### Data Analysis

QuantaSoft Analysis Pro v.1.0.596 (Bio-Rad, Pleasanton, Calif., USA) was used for droplet-cluster classification (FIGS. 2A, 2B and 2C) and Poisson function application for absolute SMN1, SMN2, and RPP30 copies. QuantaSoft also provided SMN1 and SMN2 CNV calculated as the fractional abundance of each target relative to RPP30. For the SMA [2+0] assay, QuantaSoft provided T allele and G allele copy number, as well as the fractional abundance of G alleles relative to T alleles. Rejection criteria for excluding data included (i) QX200 reader obstruction, (ii) insufficient acceptable droplets, which were <5,000 droplets, and (iii)<300 RPP30 copies/well or >10⁵ RPP30 copies/well.

Specimens were resulted as SMA positive, SMA carrier or SMA negative. Assay design and analytical precision approaches followed guidelines described elsewhere (see, e.g., CLSI document EP17-A2: Evaluation of Detection Capability for Clinical Laboratory Measurement Procedures (2012); and CLSI document EP05-A3: Evaluation of Precision of Quantitative Measurement Procedures; Approved Guideline—Third Edition (2014)).

### SMN1 and SMN2 Copy Number by Multiple Ligation-Dependent Probe Amplification (MLPA)

DdPCR was compared against MLPA to evaluate accuracy. Seventeen blood or cultured-cell samples from SMA patients, SMA carriers, and normal donors were tested using the SALSA MLPA kit P060-B2 SMA (M R C Holland, Amsterdam, Netherlands) as described elsewhere (see, e.g., Arkblad et al., Neuromuscul Disord, 16:830-8 (2006)). This kit contained specific probes to exon 7 and exon 8 of both SMN1 and SMN2 genes. DNA fragments from MLPA were analyzed on an ABI 3130 Genetic Analyzer (Applied Biosystems, Foster City, Calif.) with GeneMarker software v2.6.7 (SoftGenetics, State College, Pa.). Only SMN1 exon 7 and SMN2 exon 7 results were compared since the ddPCR SMA assay cannot detect SMN1 exon 8 CNV.

### Statistical Analysis

The Analyze-it Method Validation Edition Software (Analyze-it, Leeds, UK) for Microsoft Excel (Microsoft, Redmond, Wash.) aided data management and statistical analyses.

### Results

SMA ddPCR Assay

Distinct droplet clusters were observed for each specimen and were classifiable for quantification using QuantaSoft (FIG. 2). The clusters corresponded to target-negative droplets (grey), SMN1+ (yellow), SMN2+ (red), RPP30+ (purple), SMN1+/SMN2+ (brown), SMN2+/RPP30+ (blue), SMN1+/RPP30+ (pink) and SMN2+/SMN1+/RPP30+ triple-positive droplets (orange) (FIG. 2A). Specimens from SMA-positive patients resulted in only four clusters as shown in FIG. 2B. These clusters consisted of negative droplets, SMN2+, RPP30+ and SMN2+/RPP30+ droplets, as SMN1+ droplets were absent. When DNA was withheld, only negative droplets were observed (FIG. 2C). A minimal number of SMN1+, SMN2+ and RPP30+ droplets were appreciated when assessing carryover during the automated analytical process, but the resulting RPP30+ copies/well were significantly below the lower limit of quantitation. The total number of droplets generated per reaction averaged 15188 droplets (range: 5179 to 21077).

### Performance Characteristics

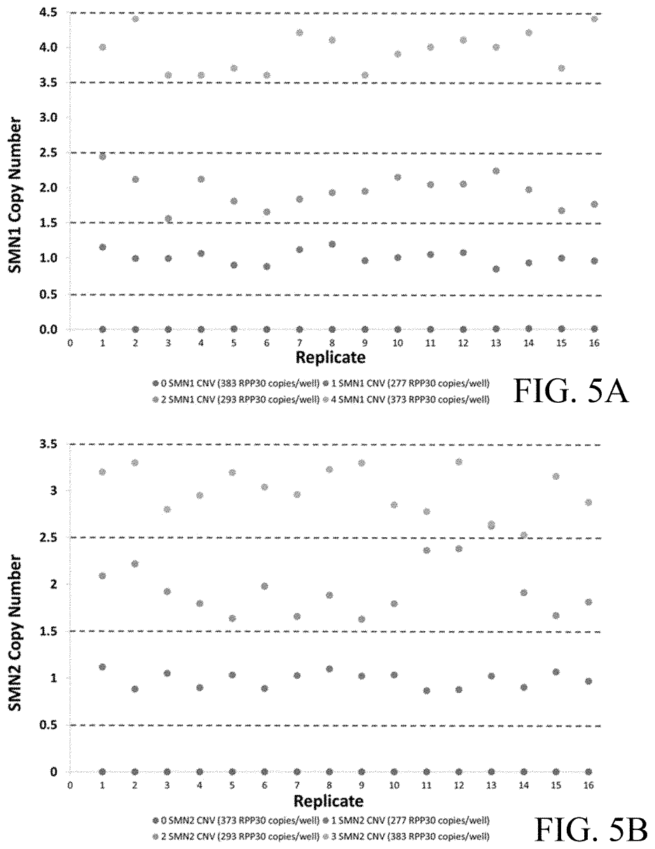

The limit of blank (LOB) was established at 1.6 RPP30 copies/well, 0.0 SMN1 copies/well, and 0.0 SMN2 copies/well, which was determined by 20 replicate measurements of blank Whatman 903 card punches. The limit of detection (LOD) was established at 8 RPP30 copies/well using the CLSI formula where LOD=LOB+1.645*SD, where SD is the standard deviation from 20 replicates of a sample with a low (11.9) RPP30 copies/well. In order to apply exact SMN1 exon 7 and SMN2 CNV levels to patient samples, we completed a study of the lower limit of CNV quantitation by diluting DNA samples with different SMN1 CNV and SMN2 CNVs to a range of RPP30 copies (277-383 RPP30 copies/well) that was above the LOD but at a concentration that would test the imprecision of the CNV calculation (FIGS. 5A and 5B). As shown in FIG. 5A, the imprecision of the CNV calculation at different CNV levels permitted limits to be set at <0.5 SMN1 CNV for homozygous SMN1 deletions, 0.5-1.4 SMN1 CNV for one SMN1 allele, 1.5-2.4 SMN1 CNV for two SMN1 alleles, 2.5-3.4 SMN1 for three SMN1 alleles, 3.5-4.4 SMN1 CNV for 4 SMN1 alleles, and 4.5-5.4 SMN1 CNV for five SMN1 alleles. The same CNV limits were observed for SMN2 CNV analysis (FIG. 5B).

### Reportable Range

The dynamic range cdPCR is extendable by dilutions or replicates to increase partitions up to infinity. A linearity study was performed and exemplified by RPP30 response with DNA from an SMA patient, a SMA carrier, and a normal donor. Two-fold serial dilution experiments on multiple samples resulted in RPP30 copies/well concentrations that fit into linear trend-lines with R² correlation coefficients >0.9984 and calculated concentrations ranging between 7 to 15958 RPP30 copies/well.

### SMN1 and SMN2 Copy Number Analysis by MLPA

MLPA was performed to assess the SMA ddPCR method accuracy. Seventeen DNA samples showed near complete concordance for SMN1 deletions with the ddPCR assay. Three samples' MLPA results could not be assigned a specific copy number because their dosage quotient fell within an “Ambiguous” range as defined by the kit insert. SMN1 results ranged from 0 to 4 CNVs while SMN2 results ranged from 0 to 5 CNVs (Table 4).

TABLE 4
SMA MLPA/ddPCR Result Comparison.

| | MLPA | MLPA | ddPCR | ddPCR |
|---|---|---|---|---|
| Sample ID | SMN1 CNV | SMN2 CNV | SMN1 CNV | SMN2 CNV |
| MLPA01 | 0 | 3 | 0.0 | 3.2 |
| MLPA02 | 0 | 3 | 0 | 3 |
| MLPA03 | 1 | 1 | 0.9 | 1 |
| MLPA04 | 1 | 1 | 0.9 | 0.9 |
| MLPA05 | 1 | 0 | 1.0 | 0.0 |
| MLPA06 | 1 | Ambiguous* | 1.1 | 2.9 |
| MLPA07 | 1 | Ambiguous* | 1.1 | 5.2 |
| MLPA08 | 2 | 1 | 1.8 | 0.9 |
| MLPA09 | 2 | 1 | 1.9 | 0.9 |
| MLPA10 | 2 | 2 | 1.9 | 1.9 |
| MLPA11 | 2 | 4 | 2.0 | 3.7 |
| MLPA12 | 2 | 0 | 2.0 | 0.0 |
| MLPA13 | 2 | 2 | 2.1 | 2.0 |
| MLPA14 | 2 | 0 | 2.2 | 0.0 |
| MLPA15 | Ambiguous* | 0 | 3.1 | 0.0 |
| MLPA16 | 4 | 0 | 4.1 | 0.0 |
| MLPA17 | 3 | 0 | 4.1 | 0.0 |

*Ambiguous copy number based on MLPA Dosage Quotient kit-insert guidelines.

### Analytical Precision

Nine DBS, whole blood, and cultured cells specimens from SMA patients, SMA carriers, and normal donors were used for intra-assay and inter-assay precision studies. Specimen were tested in triplicate for intra-assay and inter-assay replicates. Intra-assay measurements resulted with a 0% CV for SMA patients with 0 SMN1 copies (range: 0-0 copies), 6.0% CV for SMA carriers with 1 copy of SMN1 (range: 0.9-1.0 copies), and 3.0% CV for samples with 2 copies of SMN1 (range: 1.9-2.0 copies). Inter-assay measurements showed a 0% CV for SMA patients with 0 SMN1 copies (range: 0-0 copies), 7.1% CV for SMA carriers with 1 copy of SMN1 (range: 0.9-1.1 copies), and 4.0% CV for samples with 2 SMN1 copies (range: 1.9-2.2 copies).

### Clinical Validation

SMA patients contained 0 SMN1 copies and either 2 or 3 copies of SMN2 (Table 5). In this cohort, RPP30 levels correlated with DNA concentration with a median quantity of 5020 RPP30 copies/well (range: 2079 to 34627 RPP30 copies/well). A minimum lower limit of 300 RPP30 copies/well was adopted to ensure that sufficient DNA was obtained for SMN1 and SMN2 CNV detection.

TABLE 5
Clinical Validation with SMA patients.

| | | ddPCR | ddPCR |
|---|---|---|---|
| Sample ID | Sample Source | SMN1 CNV | SMN2 CNV |
| ACC01 | WB | 0 copies | 2 copies |
| ACC02 | WB | 0 copies | 2 copies |
| ACC03 | WB | 0 copies | 2 copies |
| ACC04 | WB | 0 copies | 2 copies |
| ACC05 | WB | 0 copies | 2 copies |
| ACC06 | WB | 0 copies | 2 copies |
| ACC07 | WB | 0 copies | 2 copies |
| ACC08 | WB | 0 copies | 2 copies |
| ACC09 | WB | 0 copies | 2 copies |
| ACC10 | WB | 0 copies | 3 copies |
| ACC11 | AFC | 0 copies | 2 copies |
| ACC12 | AFC | 0 copies | 2 copies |

*CNV, copy number variant; WB, whole blood; AFC, amniotic fluid culture.

### Reference Range

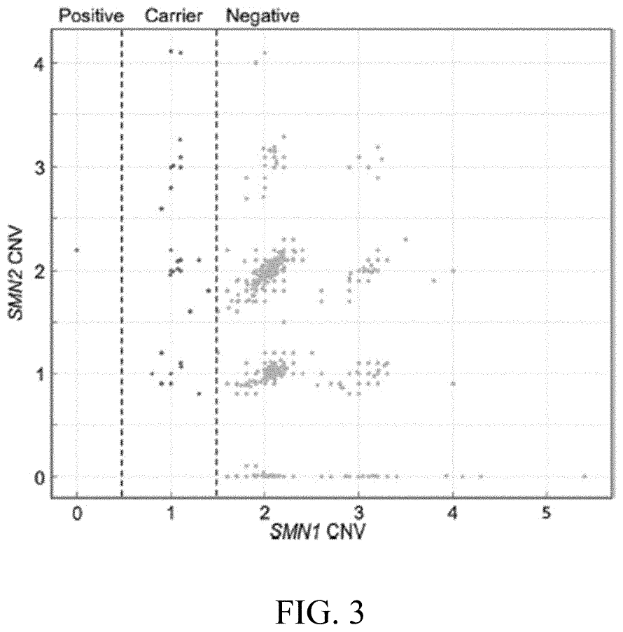

Population studies were performed for DBS and blood. SMN1 CNV ranged from 1 to 5 CNV, while SMN2 ranged from 0 to 4 CNV (Table 6, FIG. 3). The absolute RPP30 amount median was 5595 RPP30 copies/well (range: 359 to 24584 RPP30 copies/well).

TABLE 6
SMA Newborn Screening Results.

| | | Sam- | | Mean | | | | |
|---|---|---|---|---|---|---|---|---|
| | Copy | ple | | Copy | | | | |
| | num- | Num- | | Num- | | | | |
| Gene | ber | ber | % Freq | ber | Min | Max | SD | CV |
| SMN1 | 0 | 1* | 0.1% | 0.0 | — | — | — | |
| | 1 | 38 | 2% | 1.0 | 0.8 | 1.4 | 0.12 | 11% |
| | | | ( 1/40) | | | | | |
| | 2 | 1337 | 87% | 2.1 | 1.5 | 2.4 | 0.11 | 6% |
| | 3 | 144 | 9% | 3.1 | 2.5 | 3.4 | 0.18 | 6% |
| | 4 | 9 | 1% | 4.0 | 3.5 | 4.3 | 0.23 | 6% |
| | 5 | 1 | 0.1% | 5.4 | — | — | — | |
| SMN2 | 0 | 146 | 10% | 0.004 | 0.0 | 0.2 | 0.02 | 505% |
| | 1 | 586 | 39% | 1.0 | 0.8 | 1.2 | 0.06 | 6.5% |
| | 2 | 746 | 48% | 2.0 | 1.5 | 2.3 | 0.11 | 5.7% |
| | 3 | 47 | 3.2% | 3.0 | 2.6 | 3.3 | 0.15 | 4.9% |
| | 4 | 4 | 0.4% | 4.1 | 4.0 | 4.1 | 0.05 | 1.1% |

*SMA diagnosis confirmed by external laboratory findings and clinical diagnosis. SD, standard deviation; CV, coefficient of variation. Sample number total (n = 1530).

### SMN1 [2+0] Allele Detection

De-identified samples were evaluated for the SMA [2+0] allele by ddPCR. The SMN1 T allele (WT) copies, SMN1 G allele (MUT) copies, and % Fractional Abundance (% FA) of G versus T were recorded. Eleven of 125 samples (8.8%) had g.27134T>G haplotypes indicative of a SMA [2+X] allele (Tables 7 and 8). Indeed, g.27134G was found in donors with 2, 3 or 4 SMN1 CNV (Table 8), which would be suggestive of SMN1 [2+0], [2+1], or [2+2]. The specimens with 2 alleles of SMN1 (DA01 and DA02) and % FA indicating one copy of T or G apiece at position g.27134 are at risk for SMA [2+0] carrier status. All samples with the g.27134G allele were confirmed by SMN1-specific Sanger sequencing.

TABLE 7
SMA [2 + 0] Allele Assay Results.

| | Copy | Sample | | Samples with |
|---|---|---|---|---|
| Gene | Number | Count | % Freq | g.27134T > G |
| SMN1 | 1 | 3 | 2% ( 1/42) | 0 |
| | 2 | 106 | 85% | 2 |
| | 3 | 12 | 10% | 5 |
| | 4 | 4 | 3% | 4 |
| SMN2 | 0 | 10 | 8% | |
| | 1 | 59 | 47% | |
| | 2 | 53 | 42% | |
| | 3 | 3 | 2% | |

Sample number total (n = 125).

TABLE 8
Samples with g.27134T > G.

| | | | | | SMN1 MUT |
|---|---|---|---|---|---|
| Sample | SMN1 | SMN2 | SMN1 WT | SMN1 MUT | % Fractional |
| ID | CNV | CNV | (copies/μl) | (copies/μl) | Abundance |
| DA01 | 1.9 | 0.9 | 177 | 173 | 49 |
| DA02 | 2.1 | 2.0 | 95 | 94 | 50 |
| DA03 | 2.9 | 1.0 | 774 | 303 | 28 |
| DA04 | 3.0 | 0.0 | 186 | 353 | 65 |
| DA05 | 3.0 | 1.0 | 207 | 97 | 32 |
| DA06 | 3.0 | 1.0 | 309 | 154 | 33 |
| DA07 | 3.1 | 1.0 | 685 | 305 | 31 |
| DA08 | 4.0 | 0.0 | 0 | 344 | 100 |
| DA09 | 4.0 | 0.0 | 620 | 572 | 48 |
| DA10 | 4.1 | 1.0 | 578 | 178 | 24 |
| DA11 | 4.3 | 0.0 | 255 | 246 | 49 |

Sample number total (n = 11). WT, g.27134T, MUT, g.27134G.

### SMN1-Specific Sanger Sequencing Assay

Thirteen individuals referred for SMA testing had 1 copy of SMN1 as determined by the SMA ddPCR assay. This cohort included individuals with a possible family history of SMA or who had symptoms that overlapped with SMA, elucidating clinical testing. One of these patients was also discovered to be compound heterozygotic for another pathogenic variant, c.770_780dupCTGATGCTTTG by the SMN1-specific Sanger sequencing assay. Additionally, this method was able to confirm another SMN1 variant, c.91dupT, in a second SMA patient previously tested by another laboratory.

### CONCLUSION

These results demonstrate that ddPCR can be used to sensitively and specifically measure SMN1 and SMN2 copy numbers in a single DBS punch, whole blood, or cultured cells. The addition of a simultaneously measured second analyte, RPP30, enhanced results confidence without the necessity to punch multiple DBS for repeat testing due to inefficient DNA extraction or the presence of PCR inhibitors. An additional benefit of this method was the ability to measure absolute SMN1 and SMN2 copy numbers and remove reliance on the performance of a standard curve, which is integral to other published qPCR methods. High-throughput newborn screening can be achieved, for example, with the algorithm illustrated in FIG. 4.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for detecting homozygous deletion of exon 7 of SMN1 nucleic acid encoding a SMN polypeptide and detecting a genomic copy number of SMN2 nucleic acid encoding a SMN polypeptide in genomic DNA of a sample from a human, wherein said method comprises:
subjecting genomic DNA from said mammal to a digital droplet polymerase chain reaction (ddPCR), wherein said ddPCR comprises:
contacting said genomic DNA with (a) a first oligonucleotide primer pair for amplification of exon 7 of a SMN1 nucleic acid encoding a SMN polypeptide, wherein said first oligonucleotide primer pair is capable of generating a SMN1 exon 7 amplicon during said ddPCR, and (b) a first probe that can hybridize to said SMN1 exon 7 amplicon, wherein said first probe is a locked nucleic acid (LNA) oligonucleotide probe, and wherein said first probe comprises a first fluorescent label and at least one fluorescent quencher; and
contacting said genomic DNA with (a) a second oligonucleotide primer pair for amplification of SMN2 nucleic acid encoding a SMN polypeptide, wherein said second oligonucleotide primer pair is capable of generating a SMN2 amplicon, and (b) a second probe that can hybridize to said SMN2 amplicon, wherein said second probe is a LNA oligonucleotide probe, wherein said second probe comprises a second fluorescent label and at least one fluorescent quencher;
wherein said first fluorescent label and said second fluorescent label are different fluorescent labels; and
detecting a first fluorescent signal emitted when said first probe is hybridized to said SMN1 exon 7 amplicon, and detecting a second fluorescent signal emitted when said second probe is hybridized to said SMN2 amplicon.

2. The method of claim 1, wherein said mammal is a human.

3. The method of claim 1, wherein said mammal is a newborn mammal.

4. The method of claim 1, wherein said mammal is a prenatal mammal.

5. The method of claim 1, wherein said sample is a blood sample.

6. The method of claim 5, wherein said blood sample is a dried blood sample (DBS).

7. The method of claim 1, wherein said first oligonucleotide primer pair consists of an oligonucleotide primer including the nucleotide sequence set forth in SEQ ID NO:1 and an oligonucleotide primer including the nucleotide sequence set forth in SEQ ID NO:2.

8. The method of claim 1, wherein said first probe includes the nucleotide sequence set forth in SEQ ID NO:3, an internal quencher, and a terminal quencher on a 3′ end of said first probe, and wherein said first fluorescent label is on a 5′ end of said first probe.

9. The method of claim 1, wherein said second oligonucleotide primer pair consists of an oligonucleotide primer including the nucleotide sequence set forth in SEQ ID NO:4 and an oligonucleotide primer including the nucleotide sequence set forth in SEQ ID NO:5.

10. The method of claim 1, wherein said second probe includes the nucleotide sequence set forth in SEQ ID NO:6, an internal quencher, and a terminal quencher on a 3′ end of said second probe, and wherein said second fluorescent label is on a 5′ end of said second probe.

11. The method of claim 1, wherein said first fluorescent label is a fluorescein amidite (FAM), and wherein said second fluorescent label is a hexachloro-fluorescein (HEX).

12. The method of claim 1, wherein said ddPCR further comprises:
contacting said genomic DNA with (a) a third oligonucleotide primer pair for amplification of nucleic acid encoding a reference polypeptide, wherein said third oligonucleotide primer pair is capable of generating a reference amplicon, and (b) a third probe that can hybridize to said reference amplicon, wherein said third probe is a LNA oligonucleotide probe, wherein said third probe comprises a third fluorescent label and at least one fluorescent quencher;
wherein said third fluorescent label is different from said first fluorescent label and from said second fluorescent label; and
detecting a third fluorescent signal emitted when said third probe is hybridized to said reference amplicon.

13. The method of claim 12, wherein said third oligonucleotide primer pair consists of an oligonucleotide primer including the nucleotide sequence set forth in SEQ ID NO:7 and an oligonucleotide primer including the nucleotide sequence set forth in SEQ ID NO:8.

14. The method of claim 12, wherein said third probe includes the nucleotide sequence set forth in SEQ ID NO:9, an internal quencher, and a terminal quencher on a 3′ end of said third probe, and wherein said third fluorescent label is on a 5′ end of said third probe.

15. The method of claim 12, wherein said third fluorescent label is a HEX.

16. A method for treating a mammal having, or at risk of developing, spinal muscular atrophy (SMA), said method comprising:
detecting homozygous deletion of exon 7 of SMN1 nucleic acid encoding a SMN polypeptide and detecting a genomic copy number of SMN2 nucleic acid encoding a SMN polypeptide in genomic DNA in a sample from a human, wherein said detecting comprises:
subjecting genomic DNA from said mammal to a ddPCR, wherein said ddPCR comprises:
contacting said genomic DNA with (a) a first oligonucleotide primer pair for amplification of exon 7 of a SMN1 nucleic acid encoding a SMN polypeptide, wherein said first oligonucleotide primer pair is capable of generating a SMN1 exon 7 amplicon, and (b) a first probe that can hybridize to said SMN1 exon 7 amplicon, wherein said first probe is a LNA oligonucleotide probe, and wherein said first probe comprises a first fluorescent label and at least one fluorescent quencher; and
contacting said genomic DNA with (a) a second oligonucleotide primer pair for amplification of SMN2 nucleic acid encoding a SMN polypeptide, wherein said second oligonucleotide primer pair is capable of generating a SMN2 amplicon, and (b) a second probe that can hybridize to said SMN2 amplicon, wherein said second probe is a LNA oligonucleotide probe, wherein said second probe comprises a second fluorescent label and at least one fluorescent quencher;
wherein said first fluorescent label and said second fluorescent label are different fluorescent labels; and
detecting a first fluorescent signal emitted when said first probe is hybridized to said SMN1 exon 7 amplicon, and detecting a second fluorescent signal emitted when said second probe is hybridized to said SMN2 amplicon; and
when homozygous deletion of exon 7 of SMN1 nucleic acid encoding a SMN polypeptide is detected and when the genomic copy number of SMN2 nucleic acid encoding a SMN polypeptide is 1 or 2, administering one or more SMA treatments to said mammal.

17. The method of claim 16, wherein said SMA treatment is selected from the group consisting of administration of an agent that can increase exon 7 inclusion in SMN2 messenger ribonucleic acid (mRNA), occupational therapy, physical therapy, and speech and language therapy.

18. The method of claim 16, wherein said mammal is a human.

19. The method of claim 16, wherein said mammal is a newborn mammal.

20. The method of claim 16, wherein said mammal is a prenatal mammal.

21. The method of claim 16, wherein said sample is a blood sample.

22. The method of claim 21, wherein said blood sample is a DBS.


SEQUENCE LISTING (36 sequences)

SEQ ID NO: 1 - DNA, 34 bp, Artificial
  oligonucleotide primer
tccatataaagctatctatatatagctatctatg

SEQ ID NO: 2 - DNA, 19 bp, Artificial
  oligonucleotide primer
tgtgagcaccttccttctt

SEQ ID NO: 3 - DNA, 10 bp, Artificial
  oligonucleotide probe
tctgaaaccc

SEQ ID NO: 4 - DNA, 34 bp, Artificial
  oligonucleotide primer
tccatataaagctatctatatatagctatctata

SEQ ID NO: 5 - DNA, 19 bp, Artificial
  oligonucleotide primer
tgtgagcaccttccttctt

SEQ ID NO: 6 - DNA, 13 bp, Artificial
  oligonucleotide probe
tgtctaaaaccct

SEQ ID NO: 7 - DNA, 19 bp, Artificial
  oligonucleotide primer
agatttggacctgcgagcg

SEQ ID NO: 8 - DNA, 20 bp, Artificial
  oligonucleotide primer
gagcggctgtctccacaagt

SEQ ID NO: 9 - DNA, 23 bp, Artificial
  oligonucleotide primer
ttctgacctgaaggctctgcgcg

SEQ ID NO: 10 - DNA, 25 bp, Artificial
  oligonucleotide primer
tcctttattttccttacagggtttc

SEQ ID NO: 11 - DNA, 29 bp, Artificial
  oligonucleotide primer
attgttttacattaacctttcaacttttt

SEQ ID NO: 12 - DNA, 18 bp, Artificial
  oligonucleotide probe
ttgaacatttaaaaagtt

SEQ ID NO: 13 - DNA, 25 bp, Artificial
  oligonucleotide primer
tcctttattttccttacagggtttc

SEQ ID NO: 14 - DNA, 29 bp, Artificial
  oligonucleotide primer
attgttttacattaacctttcaacttttt

SEQ ID NO: 15 - DNA, 16 bp, Artificial
  oligonucleotide probe
tgaacagttaaaaagt

SEQ ID NO: 16 - DNA, 12 bp, Artificial
  oligonucleotide probe
gggttttagaca

SEQ ID NO: 17 - DNA, 20 bp, Artificial
  oligonucleotide primer
tgtgtggattaagatgactc

SEQ ID NO: 18 - DNA, 21 bp, Artificial
  oligonucleotide primer
gaaagtatgtttcttccacat

SEQ ID NO: 19 - DNA, 22 bp, Artificial
  oligonucleotide primer
agaagttactacaagcggtcct

SEQ ID NO: 20 - DNA, 20 bp, Artificial
  oligonucleotide primer
cacaactccagtgagcggat

SEQ ID NO: 21 - DNA, 22 bp, Artificial
  oligonucleotide primer
ggattaagatgactcttggtac

SEQ ID NO: 22 - DNA, 23 bp, Artificial
  oligonucleotide primer
ggaggatatcacctgatttaact

SEQ ID NO: 23 - DNA, 18 bp, Artificial
  oligonucleotide primer
gagccttgagactagctt

SEQ ID NO: 24 - DNA, 24 bp, Artificial
  oligonucleotide primer
aagtatatgtcaatagaaacactg

SEQ ID NO: 25 - DNA, 21 bp, Artificial
  oligonucleotide primer
ctccccactgatcaaaacgag

SEQ ID NO: 26 - DNA, 23 bp, Artificial
  oligonucleotide primer
ttttttttgtatccttacctctt

SEQ ID NO: 27 - DNA, 21 bp, Artificial
  oligonucleotide primer
agatgatagtttgccctcttc

SEQ ID NO: 28 - DNA, 21 bp, Artificial
  oligonucleotide primer
ttttttgtatccttacctctt

SEQ ID NO: 29 - DNA, 21 bp, Artificial
  oligonucleotide primer
ttcaatttctggaagcagaga

SEQ ID NO: 30 - DNA, 20 bp, Artificial
  oligonucleotide primer
caaaagtttcatgggagagc

SEQ ID NO: 31 - DNA, 23 bp, Artificial
  oligonucleotide primer
atttattaggtaatcaccactct

SEQ ID NO: 32 - DNA, 21 bp, Artificial
  oligonucleotide primer
ttctacccattagaatctggc

SEQ ID NO: 33 - DNA, 21 bp, Artificial
  oligonucleotide primer
tgggcaacatagcaagacctc

SEQ ID NO: 34 - DNA, 24 bp, Artificial
  oligonucleotide primer
acaatgcaagagtaatttaagcct

SEQ ID NO: 35 - DNA, 25 bp, Artificial
  oligonucleotide primer
agactatcaacttaatttctgatca

SEQ ID NO: 36 - DNA, 23 bp, Artificial
  oligonucleotide primer
aagtatgagaattctagtaggga